(19) United States
(12) Patent Application Publication
(10) Pub. No.: US 2025/0196025 A1
(43) Pub. Date: Jun. 19, 2025

(54) ALKALI-STABILIZED KAPPA LIGHT CHAIN-BINDING SEPARATION MATRIX

(71) Applicant: Cytiva BioProcess R&D AB, Uppsala (SE)

(72) Inventors: Ronnie Palmgren, Uppsala (SE); Jesper Hansson, Uppsala (SE); Tomas Björkman, Uppsala (SE); Gustav Rodrigo, Uppsala (SE); Linda Cato, Uppsala (SE)

(21) Appl. No.: 18/844,338

(22) PCT Filed: Mar. 8, 2023
(86) PCT No.: PCT/EP2023/055819

§ 371 Date: Sep. 5, 2024

(30) Foreign Application Priority Data
Mar. 16, 2022 (GB) 2203640.4

(51) Int. Cl. B01D 15/38; C07K 1/22

(52) U.S. Cl. CPC B01D 15/3809; C07K 1/22

(57) ABSTRACT

The present invention relates to a separation matrix for affinity chromatography and separation of biomolecules based on the presence of a kappa light chain. More specifically, the present invention relates to a separation matrix comprising at least 12 mg/ml kappa light chain-binding ligands covalently coupled to a porous support, wherein said kappa light chain-binding ligands comprise, consists essentially of, or consists of multimers of alkali-stabilized Finegoldia magna (formerly Peptostreptococcus magnus) Protein L domains; and wherein said porous support comprises polymer particles having a Dry solids weight (D_{W}) of 50-200 mg/ml, a volume-weighted median diameter (D50v) of 30-100 μm. The invention also relates to methods of using said separation matrix.

### CROSS REFERENCE TO RELATED APPLICATIONS

This application claims the priority benefit of PCT/EP2023/055819, filed on Mar. 8, 2023, which claims the priority benefit of GB Application No. 2203640.4, filed Mar. 16, 2022, the entire contents of which are incorporated herein by reference.

### SEQUENCE LISTING

The instant application contains a Sequence Listing which has been submitted electronically in ST.26 XML format and is hereby incorporated by reference in its entirety. Said ST.26 XML copy, created on Feb. 20, 2023, is named Sequence_Listing.xml and is 48,000 bytes in size.

### TECHNICAL FIELD

The present invention relates to the field of separation of biomolecules. More specifically, it relates to a separation matrix for affinity chromatography and separation of biomolecules based on the presence of a kappa light chain, such as immunoglobulins and immunoglobulin fractions. The invention also relates to methods of using said separation matrix.

### BACKGROUND

Immunoglobulins and immunoglobulin fragments represent the most prevalent biopharmaceutical products in either manufacture or development worldwide. The high commercial demand for, and hence value of, this particular therapeutic market has led to the emphasis being placed on pharmaceutical companies to maximize the productivity of their respective manufacturing processes whilst controlling the associated costs.

Affinity chromatography, typically on matrices comprising staphylococcal Protein A or variants thereof, is normally used as one of the key steps in the purification of intact immunoglobulin molecules. The highly selective binding of Protein A to the Fc chain of immunoglobulins provides for a generic step with very high clearance of impurities and contaminants.

For immunoglobulin fragments, or antibody fragments, such as Fab, single-chain variable fragments (scFv), bi-specific T-cell engagers (BiTEs), domain antibodies etc., which lack the Fc chain but have a subclass 1,3 or 4 kappa light chain, matrices comprising Protein L derived from Finegoldia magna (formerly Peptostreptococcus magnus) (B \dot{A}kerström, L Björck: J. Biol. Chem. 264, 19740-19746, 1989; W Kastem et al: J. Biol. Chem. 267, 12820-12825, 1992; B HK Nilson et al: J. Biol. Chem. 267, 2234-2239, 1992 and U.S. Pat. No. 6,822,075) show great promise as a purification platform providing the high selectivity needed.

Protein L matrices are commercially available as for instance Capto™ L from Cytiva™ and can be used for separation of kappa light chain-containing proteins such as intact antibodies, Fab fragments, scFv fragments, domain antibodies etc. About 75% of the antibodies produced by healthy humans have a kappa light chain and about 90% of therapeutic monoclonal antibodies and antibody fragments contain kappa light chains (Carter, P., Lazar, G. Next generation antibody drugs: pursuit of the ‘high-hanging fruit’. Nat Rev Drug Discov 17, 197-223 (2018). https://doi.org/10.1038/nrd.2017.227).

Any bioprocess chromatography application requires comprehensive attention to definite removal of impurities and/or contaminants. Such impurities and/or contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such impurities and/or contaminants from the matrix is usually performed after a first elution of the desired product in order to regenerate the matrix before subsequent use. Such removal usually involves a procedure known as cleaning-in-place (CIP), wherein agents capable of either inactivating or eluting impurities from the stationary phase are used. One such class of agents often used with chromatography media is alkaline solutions that are passed over the matrix. At present the most extensively used cleaning and sanitizing agent is NaOH, and it is desirable to use it in concentrations ranging from 0.05 up to e.g. 1 M, depending on the degree and nature of contamination and impurity. Protein L is however a rather alkali-sensitive protein compared to e.g. Protein A and only tolerates up to about 15 mM NaOH over a large number of cycles. This means that additional, less desirable cleaning solutions, e.g. urea or guanidinium salts, may also have to be used in order to ensure sufficient cleaning.

There is thus still a need in this field to obtain a separation matrix containing Protein L-derived ligands having an improved stability towards alkaline cleaning procedures.

### SUMMARY

According to a first aspect, the present disclosure provides for a separation matrix comprising at least 12 mg/ml kappa light chain-binding ligands covalently coupled to a porous support, wherein said kappa light chain-binding ligands comprise, consists essentially of, or consists of multimers of alkali-stabilized Finegoldia magna (formerly Peptostreptococcus magnus) Protein L domains; and said porous support comprises polymer particles having a Dry solids weight (D_{W}) of 50-200 mg/ml, a volume-weighted median diameter (D50v) of 30-100 μm.

The separation matrix according to the above may comprise at least 14 mg/ml kappa light chain-binding ligands, such as at least 14.5 mg/ml, at least 15 mg/ml, at least 15.5 mg/ml, at least 16 mg/ml, at least 16.5 mg/ml, at least 17 mg/ml, at least 17.5 mg/ml, at least 18 mg/ml, at least 18.5 mg/ml, or at least 19 mg/ml kappa light chain-binding ligands.

The porous support may have a D_{W} of 50-150 mg/ml, 50-120 mg/ml, 50-100 mg/ml, 50-90 mg/ml, 60-80 mg/ml, or 60-75 mg/ml, such as at least 63 mg/ml, or at least 65 mg/ml, or at least 70 mg/ml

The porous support may have a volume-weighted median diameter (D50v) of 35-90 μm, 40-80 μm, 50-70 μm, 55-70 μm, 55-67 μm, 58-70 μm, or 58-67 μm, such as at least 60 μm, or at least 62 μm.

The separation matrix according the above may have a Kd value, measured by inverse size exclusion chromatography with dextran of Mw 110 kDa as a probe molecule, of 0.6-0.95, such as a Kd value of 0.7-0.9, or a Kd value of 0.6-0.8, such as a Kd value of about 0.67, or a Kd value of about 0.72, or a Kd value of about 0.75.

The polymer particles in the separation matrix according to the above may be cross-linked.

IN the separation matric according to the above, at least two of the alkali-stabilized Protein L domains may be selected from the group comprising of functional variants of a B1 domain, a B2 domain, a B3 domain, a B4 domain, a B5 domain, a C2 domain, a C3 domain, a C4 domain and a D1 domain of Finegoldia magna (formerly Peptostreptococcus magnus) Protein L, wherein the positions which in an alignment corresponds to positions 10 and 45 in a B2 domain (SEQ ID NO 1) are histidine, and the position which in an alignment corresponds to position 60 in a B2 domain (SEQ ID NO 1) is a tyrosine or a glutamine. The at least two alkali-stabilized Protein L domains may preferably be chosen from the group comprising a B2 domain, a B3 domain, a B4 domain, a C2 domain, a C3 domain, a C4 domain and a D1 domain.

The at least two alkali-stabilized Protein L domains may have at least 90%, 95% or 98% sequence identity or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19, wherein the positions which in an alignment corresponds to positions 10 and 45 in SEQ ID NO 1, and the position which in an alignment corresponds to position 60 in SEQ ID NO 1 are not variable.

The separation matrix according to the above may comprise three, four, five, six, seven, eight or nine alkali-stabilized Protein L domains. The separation matrix according to the above may preferably comprises four, five or six alkali-stabilized Protein L domains.

The ligand may additionally comprise a coupling element, said coupling element being one or more cysteine residues, one or more lysine residues, or one or more histidine residues at the C-terminal end of the ligand. Preferably the ligand comprises one or more cysteine residues at the C-terminal end of the ligand.

Preferably, the separation matrix according to the above has a 10% breakthrough dynamic binding capacity for IgG of at least 55 mg/ml at 4 min residence time. Preferably, the separation matrix according to the above has a 10% breakthrough dynamic binding capacity for IgG of at least 70 mg/ml at 6 min residence time. Preferably, the separation matrix according to the above has a 10% breakthrough dynamic binding capacity for IgG of at least 80 mg/ml at 10 min residence time.

Preferably, the separation matrix according to the above has an IgG capacity after 24 h incubation in 0.1M NaOH at 22+/−2° C. of at least 80%, or at least 85%, or at least 90%, or at least 95% of the IgG capacity before the incubation. Preferably, the separation matrix according to any the above has an IgG capacity after 32 h incubation in 0.3M NaOH at 22+/−2° C. of at least 90% of the IgG capacity before the incubation. Preferably, the separation matrix according to the above has an IgG capacity after 12 h incubation in 0.5M NaOH at 22+/−2° C. of at least 95%, or at least 93% of the IgG capacity before the incubation.

According to another aspect the present disclosure provides for a method of isolating a kappa light chain-containing protein comprising the steps of:
a) contacting a liquid sample comprising a kappa light chain-containing protein with a separation matrix,
b) washing said separation matrix with one or a combination of several washing liquids,
c) eluting the kappa light chain-containing protein from the separation matrix with an elution liquid, and
d) cleaning the separation matrix with a cleaning liquid,
wherein the IgG capacity of the separation matrix after 24 h incubation in 0.1M NaOH at 22+/−2° C. is at least 80%, or at least 85%, or at least 90%, or at least 95% of the IgG capacity before the incubation.

According to yet another aspect, the present disclosure provides for a method for separation of bispecific antibodies comprising the steps of:
a) contacting a liquid sample comprising kappa light chain-containing proteins with a separation matrix,
b) washing said separation matrix with one or a combination of several washing liquids,
c) eluting the kappa light chain-containing protein from the separation matrix with an elution liquid and at a decreasing pH
d) cleaning the separation matrix with a cleaning liquid,
wherein the IgG capacity of the separation matrix after 24 h incubation in 0.1M NaOH at 22+/−2° C. is at least 80%, or at least 85%, or at least 90%, or at least 95% of the IgG capacity before the incubation.

Decreasing the pH in any one of the above-mentioned methods may be performed by using a pH gradient. Alternatively, decreasing the pH in any one of the above-mentioned methods may be performed in a stepwise manner. In any one of the above-mentioned methods, the decreasing pH may be from about 5.5 to about 2, such as from about 5 to about 2, from about 4.5 to about 2, or from about 4 to about 2.

The cleaning liquid may comprise 0.01-1.0 M NaOH or KOH, such as 0.05-1.0 M or 0.05-0.1 M, or 0.05-0.3M, or 0.05-0.5 NaOH or KOH.

The elution liquid may comprise at least one anion species selected from the group consisting of acetate, citrate, glycine, succinate, phosphate, and formate.

Steps a)-d) in any one of the above-mentioned methods may be repeated at least 10 times, such as at least 50 times or 50-200 times.

The separation matrix used in any one of the above-mentioned methods may be a separation matrix according to the first aspect or any variations thereof as disclosed above.

Furthermore, in yet another aspect, there is provided herein a chromatography column comprising the separation matrix according to the first aspect and any variations thereof as disclosed above.

According to a further aspect, there is provided herein the use of the chromatography column according to the above in any one of the methods disclosed above.

### FIGURES

FIG. 1: Correlation between the ligand density and alkali stability. FIG. 1a shows ligand density vs Remaining capacity after 112 CIP cycles with 0.1 M NaOH, using Trastuzumab. FIG. 1b shows DBC at 10% breakthrough (Qb10%) for different ligand densities using a Fab fragment. FIG. 1c shows the relative remaining Qb10% for a selection of ligands and Trastuzumab

FIG. 2: Illustration of the obtained model of QB10 in relation to ligand density and Kd and D_{W} for the porous support.

FIG. 3: Alkaline stability from an accelerated alkaline stability study, comparing commercially available products and different ligand densities for the prototypes. FIG. 3a shows Qb10% at 4 min residence time with Trastuzumab over 100 CIP cycles with 100 mM NaOH for two prototypes with different ligand densities and Capto™ L. FIG. 3b shows relative remaining Qb10% over 100 CIP cycles with 100 mM NaOH for two prototypes with different ligand densities and Capto™ L. FIG. 3c shows the relative remaining QB10 of a prototype for 0.1M NaOH, 0.3M NaOH and 0.5M NaOH.

FIG. 4: Qb10% vs residence time for a prototype in comparison with commercially available products: TOYOPEARL® AF-r Protein L-650F (Tosoh), Capto™ L (Cytiva™) and KanCap™ L (Kaneka). FIG. 4a shows Qb10% with Trastuzumab at different residence times. FIG. 4b shows Qb10% with Fab-fragment at different residence times. FIG. 4c shows Qb10% with dAb at different residence times

FIG. 5: Results from Chromatography with a prototype separation matrix. FIG. 5a shows a chromatogram for 50 ml bsAb01 sample, step wise elution with pH 3.4 and pH 3.1 with 5 CV elution volumes, 1 ml/min elution flow rate. FIG. 5b shows an overview of elution peaks and MS-analyzed fractions. Peak 1, Peak 2 and Peak 3 are marked with numbers 1-3. The MS analysed fractions are marked with asterisks.

FIG. 6: MS (Mass-Spectrometry) analysis of the peaks 1, 2 and 3 in FIG. 5b. FIG. 6a shows the result of non-reduced MS analysis of Peak 2 from FIG. 5b. FIG. 6b shows the result of non-reduced MS analysis of Peak 3 from FIG. 5b.

FIG. 7: Results from Chromatography with a prototype separation matrix. FIG. 7 shows a chromatogram for 10 ml BsAb01 sample, pH gradient elution chromatogram at pH 5.5-2.5 over 40 CV at 4 minutes RT. The MS analysed fractions are marked with asterisks.

FIG. 8: Overlays of Size Exclusion chromatograms for a prototype and Capto™ L. FIG. 8a shows an overlay of chromatograms for eluates. FIG. 8b shows an overlay of chromatograms for Flow-Through (FT) fractions.

### DEFINITIONS

The terms “antibody” and “immunoglobulin” may be used interchangeably herein and refers to an antigen-binding protein having a basic four-polypeptide chain structure consisting of two heavy (H) chains and two light (L) chains, said chains being stabilized by interchain or intrachain disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (VL) and a light chain constant region. The light chain constant region is comprised of one domain, CL. There are two types of light chain in humans, kappa chain and lambda chain. The term is to be understood to include any antibody, including but not limited to monoclonal antibodies and bi-specific antibodies, as well as fragments of antibodies, fusion proteins comprising antibodies or antibody fragments and conjugates comprising antibodies or antibody fragments.

The terms a “kappa light chain-binding polypeptide” and “kappa light chain-binding protein” herein mean a polypeptide or protein respectively, capable of binding to a subclass 1, 3 or 4 kappa light chain of an antibody (also called V_{κI}, V_{κIII} and V_{κIV}, as in B H K Nilson et al: J. Biol. Chem. 267, 2234-2239, 1992), and include e.g. Protein L, and any variant, fragment or fusion protein thereof that has maintained said binding property.

The term “kappa light chain-containing protein” is used as a synonym of “immunoglobulin kappa light chain-containing protein” and herein means a protein comprising a subclass 1, 3 or 4 kappa light chain (also called V_{κI}, V_{κIII} and V_{κIV}, as in B H K Nilson et al: J. Biol. Chem. 267, 10 2234-2239, 1992) derived from an antibody and includes any intact antibodies, antibody fragments, fusion proteins, conjugates or recombinant proteins containing a subclass 1, 3 or 4 kappa light chain.

The term “mAb” stands for monoclonal antibody

The term “Fab” stands for an antigen binding fragment from an immunoglobulin, comprising a kappa light chain or a lambda light chain.

The term “bi-specific antibody” stands for an antibody that can bind to two different types of antigen or two different epitopes on the same antigen. Likewise, a tri-specific antibody stands for an antibody that can bind to three different types of antigen or three different epitopes on the same antigen.

“DBC” means-Dynamic binding capacity and is the binding capacity under operating conditions, i.e., in a packed affinity chromatography column during sample application. The DBC of a chromatography resin is the amount of target protein that binds to the resin under given flow conditions before a significant breakthrough of unbound protein occurs. DBC is determined by loading a sample containing a known concentration of the target protein and monitoring the flow-through. The protein will bind to the resin to a certain break point before unbound protein will flow through the column.

The DBC can be determined on the breakthrough curve at a loss of, for example, 10% protein. This is referred to as the Qb10% value, or simply Qb10%. A sample is applied to a chromatography resin column during a specific residence time and the dynamic binding capacity for each resin is calculated at 10% of the protein breakthrough i.e., the amount of target sample that is loaded onto the column until the concentration of target sample in the column effluent is 10% of the target sample concentration in the liquid sample. If the dynamic binding capacity for each resin is calculated at 80% of the breakthrough capacity, this is referred to as the Qb80% value

Kd is a value for the fraction of the pore volume available to a probe molecule of a particular size. Herein Kd is measured by inverse size exclusion chromatography, e.g. according to the methods described in Gel Filtration Principles and Methods, Pharmacia LKB Biotechnology 1991, pp 6-15 13. with dextran of Mw 110 kDa as a probe molecule.

D50v is a value for average particle size which exceeds 50 Volume % of the particle population

The term “liquid sample” as used herein, refers to a liquid containing at least one target substance which is sought to be purified from other substances also present. Liquid samples can, for example, be aqueous solutions, organic solvent systems, or aqueous/organic solvent mixtures or solutions. The source liquids are often complex mixtures or solutions containing many biological molecules (such as proteins, antibodies, hormones, and viruses), small molecules (such as salts, sugars, lipids, etc.) and even particulate matter. While a typical source liquid of biological origin may begin as an aqueous solution or suspension, it may also contain organic solvents used in earlier separation steps such as solvent precipitations, extractions, and the like. Examples of liquid samples that may contain valuable biological substances amenable to the purification by various embodiments of the present invention include, but are not limited to, a culture supernatant from a bioreactor, a homogenized cell suspension, plasma, plasma fractions, and milk. Alternatively, the liquid sample may be referred to as “Clarified Cell Culture Feed” or “CCF”.

A “buffer” is a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. The term “physiological pH” refers to the pH of mammalian blood (i.e., 7.38 or about 7.4). Thus, a physiologic pH range is from about 7.2 to 7.6. Traditional buffer components include, but are not limited to, organic and inorganic salts, acids and bases. Exemplary buffers for use in purification of biological molecules (e.g., protein molecules) include the zwitterionic or “Good” Buffers, see e.g., Good et al. (1966) Biochemistry 5:467 and Good and Izawa (1972) Methods Enzymol. 24:62.

The “equilibration buffer” is a buffer used to prepare the binding reagent, solid phase, or both, for loading of the source liquid containing the target protein. The equilibration buffer is preferably isotonic and commonly has a pH in the range from about 6 to about 8. The “loading buffer” is a buffer used to load the source liquid, or liquid sample, containing the binding region containing protein and impurities onto the solid phase to which the binding agent is immobilized. Often, the equilibration and loading buffers are the same.

“Washing liquid” or “wash buffer” as used herein all refer herein to the liquid used to carry away impurities from the chromatography resin to which is bound the target substance. More than one wash liquid can be employed sequentially, e.g., with the successive wash liquids having varying properties such as pH, conductivity, solvent concentration, etc., designed to dissociate and remove varying types of impurities that are non-specifically associated with the chromatography resin.

“Elution liquid” or “elution buffer”, which are used interchangeably herein, refers herein to the liquid that is used to dissociate the target substance from the chromatography resin, thereby eluting the binding region-containing protein from the immobilized binding agent, after it has been washed with one or more wash liquids. The elution liquid acts to dissociate the target substance without denaturing it irreversibly. Typical elution liquids are well known in the chromatography art and may have a different pH (typically lower pH), higher concentrations of salts, free affinity ligands or analogs, or other substances that promote dissociation of the target substance from the chromatography resin. “Elution conditions” refers to process conditions imposed on the target substance-bound chromatography resin that dissociate the target substance from the chromatography resin, such as the contacting of the target substance-bound chromatography resin with an elution liquid or elution buffer to produce such dissociation.

Preferably the elution buffer has a low pH and thereby disrupts interactions between the kappa light chain binding separation matrix and the protein of interest. Preferably, the low pH elution buffer has a pH in the range from about 2 to about 5, most preferably in the range from about 3 to about 4. Examples of buffers that will control the pH within this range include glycine, phosphate, acetate, and citrate buffers, as well as combinations of these. The preferred such buffers are citrate and acetate buffers, most preferably sodium citrate or sodium acetate buffers.

Cleaning liquid may be an acidic solution or an alkali solution for removing resin residues after elution of the target substance. For instance precipitated proteins, hydrophobic proteins, nucleic acids, endotoxins and viruses may be removed by the cleaning liquid. Most commonly, alkali solutions are used for the purpose

Cleaning-in-place (CIP) is an important process for efficient use of a chromatography column. In order to maximize the number of cycles that a column can be reused, a cleaning procedure that efficiently removes impurities without being harmful to the chromatography resin is required.

“HCP” stands for Host Cell Protein.

As used herein, the terms “comprises”, “comprising”, “containing”, “having” and the like can mean “includes”, “including”, and the like; “consisting essentially of” or “consists essentially” is an open-ended term, allowing for the presence of more than that which is recited so long as basic or novel characteristics of that which is recited is not changed by the presence of more than that which is recited, but excludes prior art embodiments.

### DETAILED DESCRIPTION

The inventors had as an objective to invent a separation matrix containing Protein L-derived ligands having an improved stability towards alkaline cleaning procedures, while maintaining a satisfactory efficiency in binding capacity and flow characteristics in a chromatography setting, and preferably better than current commercially available separation matrices.

According to one aspect, the objective has been attained by providing a separation matrix comprising at least 12 mg/ml kappa light chain-binding ligands covalently coupled to a porous support, wherein said kappa light chain-binding ligands comprise, consists essentially of, or consists of multimers of alkali-stabilized Finegoldia (former Peptostreptococcus) Protein L domains; and said porous support comprises polymer particles having a Dry solids weight (Dw) of 50-200 mg/ml, a volume-weighted median diameter (D50v) of 30-100 μm.

The kappa light chain-binding ligands comprised in the separation matrix of the present invention comprises, consists essentially of, or consists of, multimers of alkali-stabilized Finegoldia Protein L domains. The Protein L domains may be any functional Protein L derived domain as long as it is alkali-stabilized. The Protein L domains are chosen from a functional variant of a B1 domain, a B2 domain, a B3 domain, a B4 domain, a B5 domain, a C2 domain, a C3 domain, a C4 domain and a D1 domain, wherein the positions which in an alignment corresponds to positions 10 and 45 in a B2 domain (SEQ ID NO 1) are histidine, and the position which in an alignment corresponds to position 60 in a B2 wt domain (SEQ ID NO 1) is a tyrosine or a glutamine. Thus, the above-mentioned positions corresponding to positions 10, 45 and 60 in a B2 wt domain (SEQ ID NO 1) are not variable within the functional Protein L domain.

(wt B2)
SEQ ID NO: 1
PKEEVTIKANLIYADGKTQTAEFKGTFEEAAAEAYRYADALKKDN
GEYTVDVADKGYTLNIKFAGKEKTPEE

Examples of such functional Protein L domains may be:

(B2: N10H, N45H, N60Y mutations)
SEQ ID NO: 2
PKEEVTIKAHLIYADGKTQTAEFKGTFEEAAAEAYRYADALKKDH
GEYTVDVADKGYTLYIKFAGKEKTPEE
(B2: N10H, N45H, N60Q mutations)
SEQ ID NO: 3
PKEEVTIKAHLIYADGKTQTAEFKGTFEEAAAEAYRYADALKKDH
GEYTVDVADKGYTLQIKFAGKEKTPEE
(B3: N10H, N45H, N60Y mutations)
SEQ ID NO: 4
PKEEVTIKAH LIYADGKTQT AEFKGTFEEA TAEAYRYADL
LAKEHGKYTV DVADKGYTLY IKFAGKEKTP EE
(B3: N10H, N45H, N60Q mutations)
SEQ ID NO: 5
PKEEVTIKAH LIYADGKTQT AEFKGTFEEA TAEAYRYADL
LAKEHGKYTV DVADKGYTLQ IKFAGKEKTP EE
(B1: N10H, N45H, N60Y mutations)
SEQ ID NO: 6
SEEEVTIKAHLIFANGSTQTAEFKGTFEKATSEAYAYADTLKKDH
GEYTVDVADKGYTLYIKFAGKEKTPEE
(B1: N10H, N45H, N60Q mutations)
SEQ ID NO: 7
SEEEVTIKAHLIFANGSTQTAEFKGTFEKATSEAYAYADTLKKDH
GEYTVDVADKGYTLQIKFAGKEKTPEE
(B4: N10H, N45H, N60Y mutations)
SEQ ID NO: 8
PKEEVTIKAHLIYADGKTQTAEFKGTFAEATAEAYRYADLLAKEH
GKYTADLEDGGYTIYIRFAGKKVDEKPEE
(B4: N10H, N45H, N60Q mutations)
SEQ ID NO: 9
PKEEVTIKAHLIYADGKTQTAEFKGTFAEATAEAYRYADLLAKEH
GKYTADLEDGGYTIQIRFAGKKVDEKPEE
(B5: N9H, N44H, N59Y mutations)
SEQ ID NO 10
KEQVTIKEHIYFEDGTVQTATFKGTFAEATAEAYRYADLLSKEHG
KYTADLEDGGYTIQIRFAGKEEPEE
(B5: N9H, N44H, N59Q mutations)
SEQ ID NO 11
KEQVTIKEHIYFEDGTVQTATFKGTFAEATAEAYRYADLLSKEHG
KYTADLEDGGYTIQIRFAGKEEPEE
(C2b N57Y: N10H, N45H, N60Y mutations)
SEQ ID NO 12
PKEEVTIKVHLIFADGKTQTAEFKGTFEEATAKAYAYADLLAKEH
GEYTADLEDGGYTIYIKFAGKETPETPEE
(C2b N57Y: N10H, N45H, N60Q mutations)
SEQ ID NO 13
PKEEVTIKVHLIFADGKTQTAEFKGTFEEATAKAYAYADLLAKEH
GEYTADLEDGGYTIQIKFAGKETPETPEE
(C3b N39D, N57Y: N10H, N45H, N60Y mutations)
SEQ ID NO 14
PKEEVTIKVHLIFADGKIQTAEFKGTFEEATAKAYAYADLLAKEH
GEYTADLEDGGYTIYIKFAGKETPETPEE
(C3b N39D, N57Y: N10H, N45H, N60Q mutations)
SEQ ID NO 15
PKEEVTIKVHLIFADGKIQTAEFKGTFEEATAKAYAYADLLAKEH
GEYTADLEDGGYTIQIKFAGKETPETPEE
(C4: N10H, N45H, N60Y mutations)
SEQ ID NO: 16
PKEEVTIKVHLIFADGKTQTAEFKGTFEEATAEAYRYADLLAKVH
GEYTADLEDGGYTIYIKFAGKEQPGENPG
SEQ ID NO: 17
(C4: N10H, N45H, N60Q mutations)
PKEEVTIKVHLIFADGKTQTAEFKGTFEEATAEAYRYADLLAKVH
GEYTADLEDGGYTIQIKFAGKEQPGENPG
(D1: N10H, N45H, N60Y mutations)
SEQ ID NO: 18
PKEEVTIKAHLIFADGKTQTAEFKGTFEEATAEAYRYADLLAKVH
GEYTADLEDGGYTIYIKFAGKEQPGEN
(D1: N10H, N45H, N60Q mutations)
SEQ ID NO: 19
PKEEVTIKAHLIFADGKTQTAEFKGTFEEATAEAYRYADLLAKVH
GEYTADLEDGGYTIQIKFAGKEQPGEN

Preferably, the Protein L domain is selected from the group comprising of the B3 domain, the C2 domain, the C3 domain and the D-domain, wherein the positions which in an alignment corresponds to positions 10 and 45 in a B2 wt domain (SEQ ID NO 1) are histidine, and the position which in an alignment corresponds to position 60 in a B2 wt domain (SEQ ID NO 1) is a tyrosine or a glutamine. The above-mentioned positions corresponding to positions 10, 45 and 60 in a B2 wt domain (SEQ ID NO 1) are not variable within the functional Protein L domain.

The remaining positions in such a functional Protein L domain may be varied as long as the three-dimensional structure is not altered as compared to that of the B2 wt domain (SEQ ID NO 1), and as long as it at least retains the kappa light chain-binding capacity and is alkali-stabilized as compared to the B2 wt domain (SEQ ID NO 1). The variation may be conservative amino acid substitutions for an amino acid with a similar or identical charge, hydrophobicity, etc., and the skilled person is able to determine what such a variation of an amino acid may be.

The Protein L domain may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19.

The functional Protein L domain may be a truncated sequence. For instance the positions corresponding to positions 1-4 in B2 wt domain (SEQ ID NO 1) may be deleted. For instance positions corresponding to positions following position 65 in B2 wt domain (SEQ ID NO 1) may be deleted.

(B1_trunc, N6H, N41H, N56Y mutations)
SEQ ID NO: 20
VTIKAHLIFANGSTQTAEFKGTFEKATSEAYAYADTLKKDHGEYT
VDVADKGYTLYIKFAG
(B1_trunc, N6H, N41H, N56Q mutations)
SEQ ID NO: 21
VTIKAHLIFANGSTQTAEFKGTFEKATSEAYAYADTLKKDHGEYT
VDVADKGYTLQIKFAG
(B2_trunc, N6H, N41H, N56Y mutations)
SEQ ID NO: 22
VTIKAHLIYADGKTQTAEFKGTFEEAAAEAYRYADALKKDHGEYT
VDVADKGYTLYIKFAG
(B2_trunc, N6H, N41H, N56Q mutations)
SEQ ID NO: 23
VTIKAHLIYADGKTQTAEFKGTFEEAAAEAYRYADALKKDHGEYT
VDVADKGYTLQIKFAG
(B3_trunc, N6H, N41H, N56Y mutations)
SEQ ID NO: 24
VTIKAHLIYADGKTQTAEFKGTFEEATAEAYRYADLLAKEHGKYT
VDVADKGYTLYIKFAG
(B3_trunc, N6H, N41H, N56Q mutations)
SEQ ID NO: 25
VTIKAHLIYADGKTQTAEFKGTFEEATAEAYRYADLLAKEHGKYT
VDVADKGYTLQIKFAG
(B4_trunc, N6H, N41H, N56Y mutations)
SEQ ID NO: 26
VTIKAHLIYADGKTQTAEFKGTFAEATAEAYRYADLLAKEHGKYT
ADLEDGGYTIYIRFAG
(B4_trunc, N6H, N41H, N56Q mutations)
SEQ ID NO: 27
VTIKAHLIYADGKTQTAEFKGTFAEATAEAYRYADLLAKEHGKYT
ADLEDGGYTIQIRFAG
(B5_trunc, N6H, N41H, N56Y mutations)
SEQ ID NO: 28
VTIKEHIYFEDGTVQTATFKGTFAEATAEAYRYADLLSKEHGKYT
ADLEDGGYTIYIRFAG
(B5_trunc, N6H, N41H, N56Q mutations)
SEQ ID NO: 29
VTIKEHIYFEDGTVQTATFKGTFAEATAEAYRYADLLSKEHGKYT
ADLEDGGYTIQIRFAG
(C2b N57Y _trunc, N6H, N41H, N56Y mutations)
SEQ ID NO: 30
VTIKVHLIFADGKTQTAEFKGTFEEATAKAYAYADLLAKEHGEYT
ADLEDGGYTIYIKFAG
(C2b N57Y _trunc, N6H, N41H, N56Q mutations)
SEQ ID NO: 31
VTIKVHLIFADGKTQTAEFKGTFEEATAKAYAYADLLAKEHGEYT
ADLEDGGYTIQIKFAG
(C3b N39D, N57Y trunc, N6H, N41H, N56Y mutations)
SEQ ID NO: 32
VTIKVHLIFADGKIQTAEFKGTFEEATAKAYAYADLLAKEHGEYT
ADLEDGGYTIYIKFAG
(C3b N39D, N57Y_trunc, N6H, N41H, N56Q mutations)
SEQ ID NO: 33
VTIKVHLIFADGKIQTAEFKGTFEEATAKAYAYADLLAKEHGEYT
ADLEDGGYTIQIKFAG
(C4_trunc, N6H, N41H, N56Y mutations)
SEQ ID NO: 34
VTIKVHLIFADGKTQTAEFKGTFEEATAEAYRYADLLAKVHGEYT
ADLEDGGYTIYIKFAG
(C4_trunc, N6H, N41H, N56Q mutations)
SEQ ID NO: 35
VTIKVHLIFADGKTQTAEFKGTFEEATAEAYRYADLLAKVHGEYT
ADLEDGGYTIQIKFAG
(D1_trunc, N6H, N41H, N56Y mutations)
SEQ ID NO: 36
VTIKAHLIFADGKTQTAEFKGTFEEATAEAYRYADLLAKVHGEYT
ADLEDGGYTIYIKFAG
(D1_trunc, N6H, N41H, N56Q mutations)
SEQ ID NO: 37
VTIKAHLIFADGKTQTAEFKGTFEEATAEAYRYADLLAKVHGEYT
ADLEDGGYTIQIKFAG

The Protein L domain may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:20, SEQ ID NO:21, SEQ ID NO:22, SEQ ID NO: 23, SEQ ID NO: 24, SEQ ID NO: 25, SEQ ID NO: 26, SEQ ID NO: 27, SEQ ID NO:28, SEQ ID NO: 29, SEQ ID NO: 30, SEQ ID NO:31, SEQ ID NO: 32, SEQ ID NO:33, SEQ ID NO:34, SEQ ID NO:35, SEQ ID NO: 36 or SEQ ID NO:37.

As can be seen above, the C2-domain and the C₃ domain comprise additional mutation(s). The C2 domain scaffold comprises an additional N57Y mutation and is herein named C2b. The C3 domain scaffold comprises additional N39D and N57Y mutations and is herein named C3b.

As specified above, the kappa light chain-binding ligands comprised in the separation matrix comprise, consists essentially of, or consists of multimers of the alkali-stabilized Protein L domains. The multimer may comprise two, three, four, five, six, seven, eight or nine alkali-stabilized Protein L domains. In an alternative language, the multimers may be a dimer, trimer, tetramer, pentamer, hexamer, heptamer, octamer or a nonamer. Preferably, the ligands comprise four, five, six or seven alkali-stabilized Protein L domains, such as five or six alkali-stabilized Protein L domains.

The multimer may further comprise a linker, spacer, or additional amino acid(s). The additional amino acid(s) may for instance originate from the cloning process of the ligand or constitute a residue from a cleaved off signaling sequence.

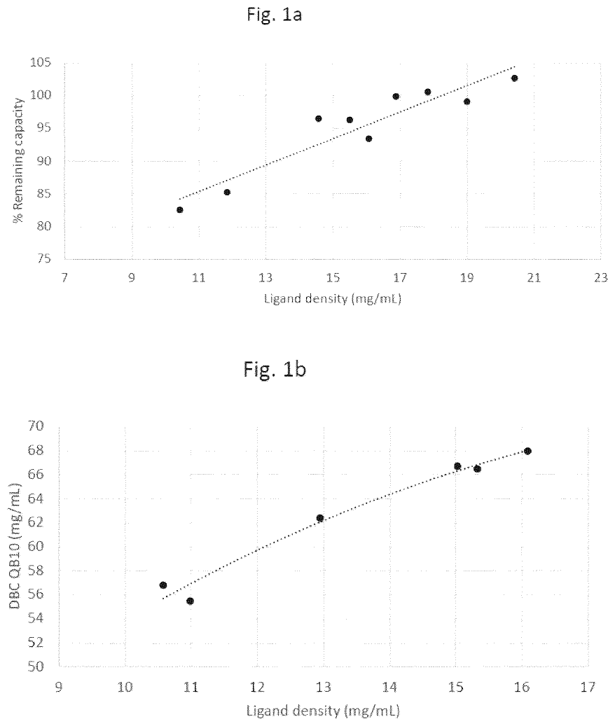

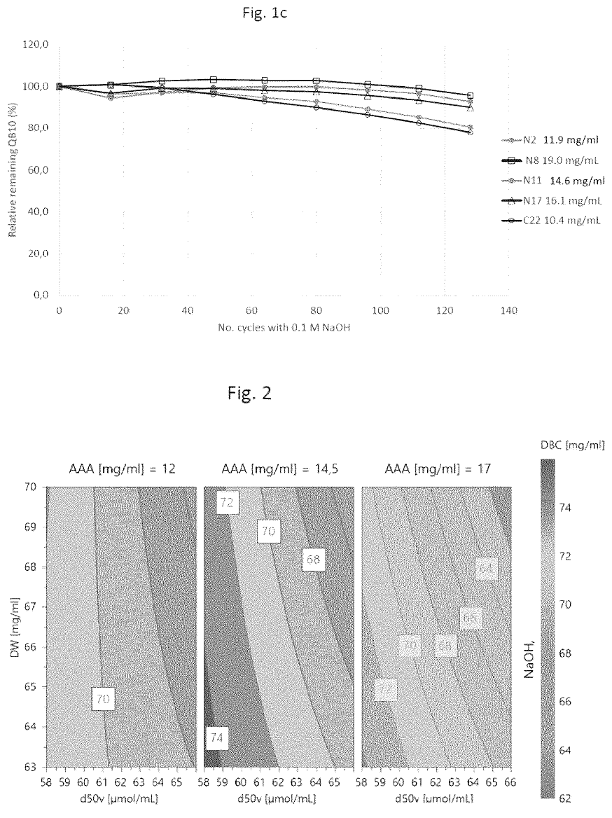

As specified above, the separation matrix comprises at least 12 mg/ml kappa light chain-binding ligands. Preferably, the separation matrix comprises at least 14 mg/ml kappa light chain-binding ligands, such as at least 14.5 mg/ml, at least 15 mg/ml, at least 15.5 mg/ml, at least 16 mg/ml, at least 16.5 mg/ml, at least 17 mg/ml, at least 17.5 mg/ml, at least 18 mg/ml, at least 18.5 mg/ml or at least 19 mg/ml kappa light chain-binding ligands. FIG. 1a shows the correlation between the ligand density and alkali stability in terms of % remaining capacity of the separation matrix for Trastuzumab. FIG. 1b shows the correlation between ligand density and Qb10% for Fab. From both of these FIGS. 1a and 1b, it is clear that a ligand density of at least 12 mg/ml is advantageous for both alkali stability and Qb10%.

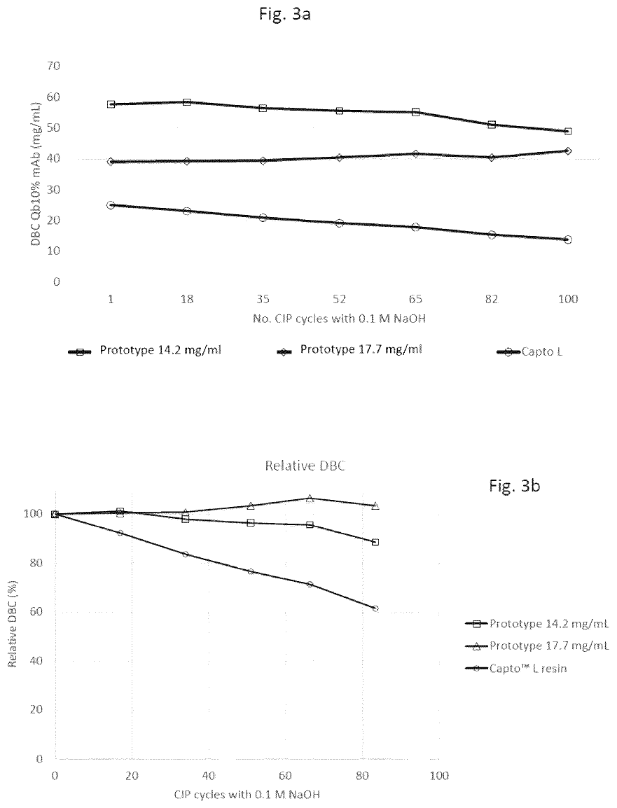

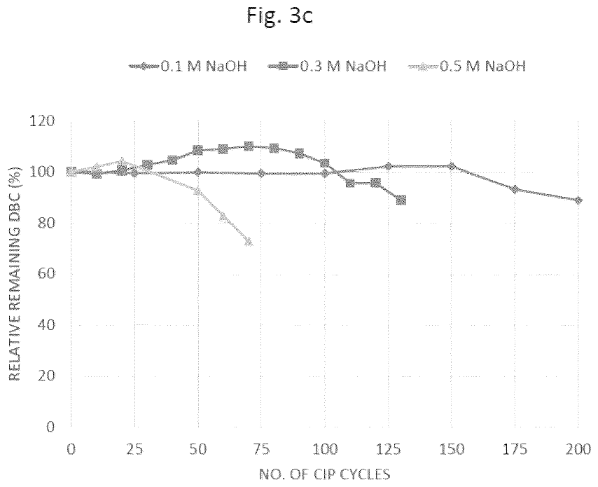

Additionally, as shown in the Examples below, these preferred ligand densities provides for an improved Qb10% as well as an improved alkaline stability compared to existing product on the market, further illustrated in FIGS. 3a and 3b.

The amount of coupled polypeptide/multimer can be controlled by the concentration of polypeptide/multimer used in the coupling process, by the coupling conditions used and/or by the pore structure of the support used. As a general rule the absolute binding capacity of the matrix increases with the amount of coupled polypeptide/multimer, at least up to a point where the pores become significantly constricted by the coupled polypeptide/multimer. The relative binding capacity per mg coupled polypeptide/multimer will decrease at high coupling levels, resulting in a cost-benefit optimum within the ranges specified above.

The ligand may comprise a coupling element, said coupling element being one or more cysteine residues, one or more lysine residues, or one or more histidine residues at the C-terminal end of the ligand. Preferably, the ligand may comprise one or more cysteine residues at the C-terminal end of the ligand.

The coupling element(s) may be directly linked to the C- or N-terminus, or it/they may be linked via a linker comprising up to 15 amino acids, such as 1-5, 1-10 or 5-10 amino acids. This stretch should preferably also be sufficiently stable in alkaline environments so as to not impair the properties of the mutated ligand. For this purpose, it is advantageous if the stretch does not contain asparagine. It can additionally be advantageous if the stretch does not contain glutamine. An advantage of having a C- or N-terminal cysteine is that endpoint coupling of the protein can be achieved through reaction of the cysteine thiol with an electrophilic group on a support, as described above. This provides excellent mobility of the coupled protein which is important for the binding capacity.

The ligand or multimers may be coupled to the support via thioether bonds. Methods for performing such coupling are well-known in this field and easily performed by the skilled person in this field using standard techniques and equipment. Thioether bonds are flexible and stable and generally suited for use in affinity chromatography. In particular when the thioether bond is via a terminal or near-terminal cysteine residue on the ligand or multimer, the mobility of the coupled ligand/multimer is enhanced which provides improved binding capacity and binding kinetics. In some embodiments the ligand/multimer is coupled via a C-terminal cysteine. This allows for efficient coupling of the cysteine thiol to electrophilic groups, e.g. epoxide groups, halohydrin groups etc. on a support, resulting in a thioether bridge coupling. The ligand/multimer can e.g. be coupled via single-point attachment, e.g. via a single cysteine or by directed multipoint attachment, using e.g. a plurality of lysines or other coupling groups near a terminus of the polypeptide/multimer.

The solid support of the matrix according to the invention can be of any suitable well-known kind. A conventional affinity separation matrix is often of organic nature and based on polymers that expose a hydrophilic surface to the aqueous media used, i.e. expose hydroxy (—OH), carboxy (—COOH), carboxamido (—CONH2, possibly in N-substituted forms), amino (—NH2, possibly in substituted form), oligo- or polyethylenoxy groups on their external and, if present, also on internal surfaces. The solid support should be porous. The porosity can be expressed as a Kav or Kd value (the fraction of the pore volume available to a probe molecule of a particular size) measured by inverse size exclusion chromatography, e.g. according to the methods described in Gel Filtration Principles and Methods, Pharmacia LKB Biotechnology 1991, pp 6-15 13. By definition, both Kd and Kav values always lie within the range 0-1. In one embodiment, the porous support has a Kd value of 0.6-0.95, such as a Kd value of 0.7-0.90, or a Kd value of 0.6-0.8, as measured with dextran of Mw 110 kDa as a probe molecule. The porous support may preferably have a Kd value of about 0.67. The porous support may preferably have a Kd value of about 0.72. The porous support may preferably have a Kd value of about 0.75.

The higher the Kd value is, the larger the pores in the porous support, and the larger the fraction of the inner volume of the beads that is accessible to a solute molecule, such as biomolecules, such as immunoglobulins. The skilled person is aware of this, and able to calculate the Kd of a porous support as described above. With the larger pores mentioned as preferred above, a larger amount of ligands are coupled to the porous support. Furthermore, with larger pores, such as mentioned above, the kappa light chain-containing protein may access the ligands also within the pores. Thus, a larger binding capacity is achieved.

Multimeric ligands, such as a pentamer or a hexamer gives higher DBC compared to lower multimeric ligands such as tetramer ligands or lower. This effect is especially true on solid supports with high Kd 0.7-0.9.

In certain embodiments the support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups, and they are generally stable towards alkaline cleaning solutions used in bioprocessing.

In some embodiments the support comprises agar or agarose. The supports used in the present invention can easily be prepared according to standard methods, such as inverse suspension gelation (S Hjerten: Biochim Biophys Acta 79 (2), 393-398 (1964)). Alternatively, the base matrices are commercially available products, such as crosslinked agarose beads sold under the name of SEPHAROSE™ FF (Cytiva™). In an embodiment, which is especially advantageous for large-scale separations, the support has been adapted to increase its rigidity using the methods described in U.S. Pat. No. 6,602,990 or U.S. Pat. No. 7,396,467, which are hereby incorporated by reference in their entirety, and hence renders the matrix more suitable for high flow rates.

In certain embodiments the support, such as a polysaccharide or agarose support, is crosslinked, such as with hydroxyalkyl ether crosslinks. Crosslinker reagents producing such crosslinks can be e.g. epihalohydrins like epichlorohydrin, diepoxides like butanediol diglycidyl ether, allylating reagents like allyl halides or allyl glycidyl ether. Crosslinking is beneficial for the rigidity of the support and improves the chemical stability. Hydroxyalkyl ether crosslinks are alkali stable and do not cause significant nonspecific adsorption.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L′Industria 70 (9), 70-75 (1988)). Alternatively, a commercially available product, such as SOURCE™ (Cytiva™) is used. In another alternative, the solid support according to the invention comprises a support of inorganic nature, e.g. silica, zirconium oxide etc. In another alternative, the support particles are magnetic. One example of such support particles is polysaccharide or synthetic polymer beads comprising e.g. magnetite particles, such that the beads can be used in magnetic batch separations.

The separation matrix is preferably in beaded or particle form that is porous. Matrices in beaded or particle form can be used as a packed bed or in a suspended form. Suspended forms include those known as expanded beds and pure suspensions, in which the particles or beads are free to move. In case of packed bed and expanded beds, the separation procedure commonly follows conventional chromatography with a concentration gradient. In case of pure suspension, batch-wise mode will be used. Preferably, the separation matrix according to the above is packed in a chromatography column.

A resin designed for large scale chromatography and bioprocess may, in general, have a D50V typically from 30 μm up to 100 μm and a DW from 50 mg/ml to 200 mg/mL.

The flow rate should preferably be about 250-500 cm/h in large scale columns with a 20 cm bed height at a back pressure of <3 bar and with a bed volume of >3 L. The skilled person within the technical field is able to calculate the flow rate in a column of another size, height and volume, and subsequently able to adjust the settings accordingly.

The porous support in a beaded or particle form according to the present disclosure has a Dry solids weight (Dw) of 50-200 mg/ml, such as 50-150 mg/ml, 50-120 mg/ml, 50-100 mg/ml, 50-90 mg/ml, 60-80 mg/ml, or 60-75 mg/ml. Preferably, the Dw is of at least 63 mg/ml, or at least 65 mg/ml. The Dw may be at least of 70 mg/ml.

The porous support in a beaded or particle form may have a volume-weighted median diameter (D50v) of 30-100. According to the present disclosure, the D50v is preferably 35-90 μm, 40-80 μm, or 50-70 μm, such as 55-70 μm, 55-67 μm, 58-70 μm, or 58-67 μm. The D50v may for instance be at least 60 μm, or at least 62μ m.

The combination of the above-mentioned ranges for Dw and d50v allows for a high DBC to be reached. In particular the combination of the above-mentioned ranges for Dw and d50v and the above-mentioned ligand densities, allow for a high Qb10% to be reached, as shown in FIG. 2. This DBC will be reachable with a flow rate and backpressure that is still relevant for large scale chromatography as disclosed above.

FIG. 2 shows the contour-plot for the prototype model at three different ligand densities. From these plots it is evident that the upper right corner of the window will result in a lower binding capacity. This is especially true in cases when a high ligand density is obtained.

The alkali stability of the matrix can be assessed by measuring the kappa light chain-binding capacity, using e.g. a specific kappa light chain-containing protein or polyclonal human IgG, before and after incubation in alkaline solutions at a specified temperature, e.g. 22+/−2° C. The incubation can e.g. be performed in 0.1 M NaOH for a number of 15 min cycles, such as 100, 200 or 300 cycles. The binding capacity of the matrix after 100 15 min incubation cycles in 0.1 M NaOH at 22+/−2° C. can be at least 80%, such as at least 85%, at least 90% or at least 95% of the binding capacity before the incubation. Alternatively, the incubation can be performed in 0.1 M NaOH for a number of 4 h cycles, such as 6 cycles giving a total incubation time of 24 h. The binding capacity of the matrix after 24 h min total incubation time in 0.1 M NaOH at 22+/−2° C. can be at least 80%, such as at least 85%, at least 90% or at least 95% of the binding capacity before the incubation.

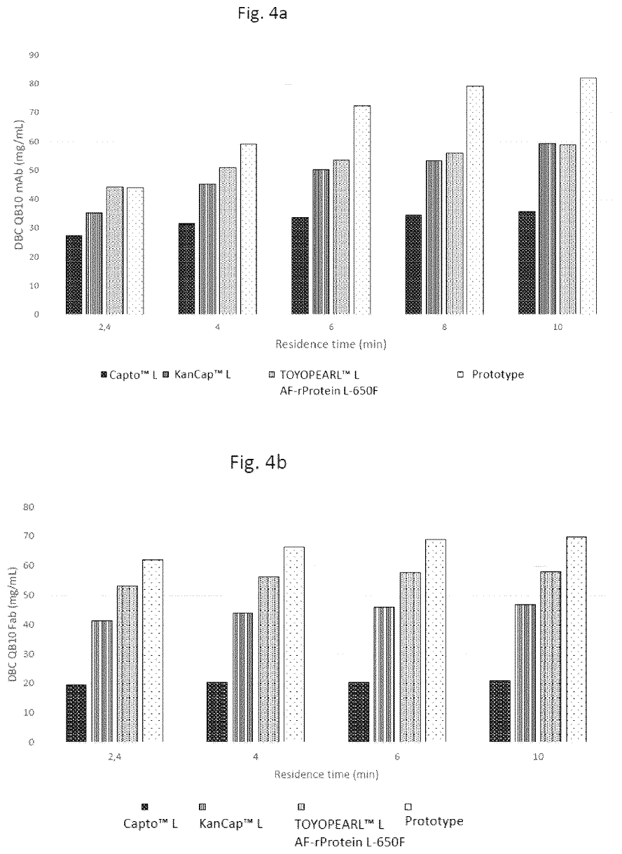

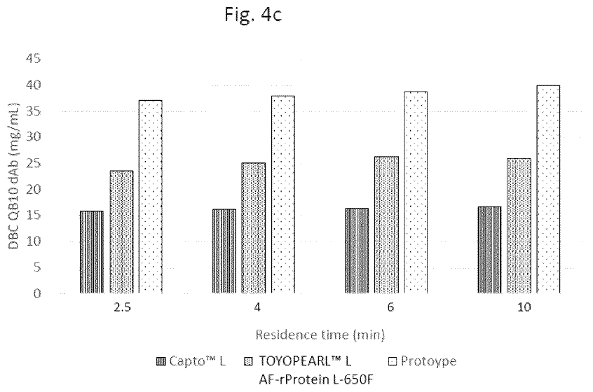

As shown in the Examples below, a separation matrix according to the present invention provides for an improved alkaline stability compared to commercially available protein L ligand resins. This is shown in for instance FIG. 4a. The separation matrix according to the above has a 10% breakthrough dynamic binding capacity for IgG of at least 55 mg/ml at 4 min residence time. Furthermore, the separation matrix according to the above has a 10% breakthrough dynamic binding capacity (Qb10%) for IgG of at least 70 mg/ml at 6 min residence time. Yet furthermore, the separation matrix according to the above has a 10% breakthrough dynamic binding capacity (Qb10%) for IgG of at least 80 mg/ml at 10 min residence time.

According to another aspect, herein is provided a method of isolating a kappa light chain-containing protein, comprising the steps of:
a) contacting a liquid sample comprising a kappa light chain-containing protein with a separation matrix,
b) washing said separation matrix with one or a combination of several washing liquids,
c) eluting the kappa light chain-containing protein from the separation matrix with an elution liquid, and
d) cleaning the separation matrix with a cleaning liquid,
wherein the IgG capacity of the separation matrix after 24 h incubation in 0.1M NaOH at 22+/−2° C. is at least 80%, or at least 85%, or at least 90%, or at least 95% of the IgG capacity before the incubation.

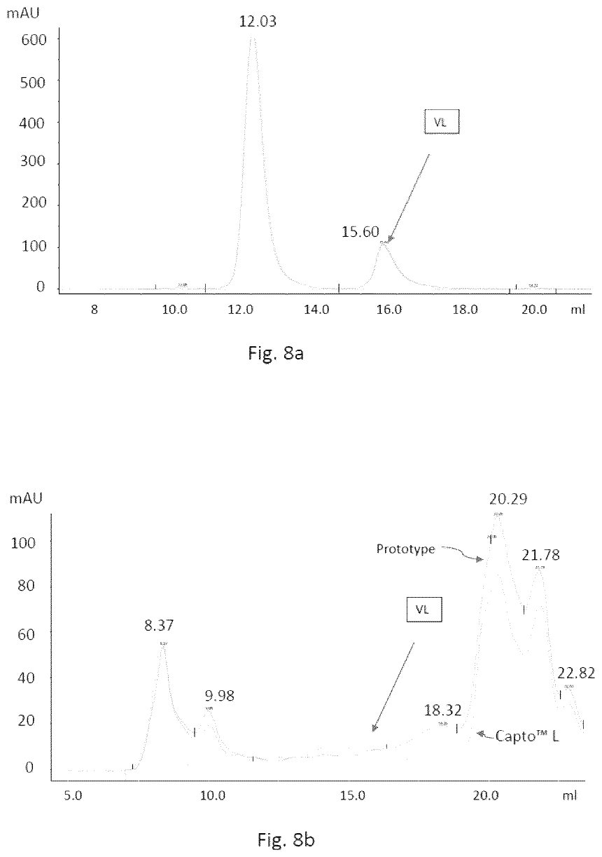

According to one embodiment, the separation matrix as disclosed above is used in said method. As can be seen in FIG. 8a, the method for isolating a kappa light chain-containing protein according to the present invention provides for a complete capture of kappa light chain (VL) from the CCF. There is no detectable presence of VL in the flow-through fraction, as shown in FIG. 8b.

The elution may be performed by using any suitable elution liquid used for elution from Protein L separation matrix. The elution liquid can e.g. be a solution or buffer with pH 4 or lower, such as pH 2.5-4 or 2.8-3.5. The elution buffer or the elution buffer gradient may comprise at least one mono-di- or trifunctional carboxylic acid or salt of such a carboxylic acid. The elution buffer or the elution buffer gradient may comprise at least one anion species selected from the group consisting of acetate, citrate, glycine, succinate, phosphate, and formate.

The generation of bispecific antibodies or IgG molecules is difficult as the pairing of the light and heavy chains, and consequently the variable domains therein (VL; VH) may be promiscuous. The pairing of two different light and two different heavy chains may lead to a large number of mispairing, as normally only one specific asymmetric combination is wanted, and a multitude of combinations achieved will be non-functional or unwanted molecules, such as for instance monospecific homodimers. Thus, there is an increasing need for improved tools and methods to separate bispecific antibodies from monospecific antibodies, as well as separating mismatched bispecific antibodies from correctly matched bispecific antibodies. This may be done by separation based on different light chains comprised in a bispecific antibody.

According to yet another aspect, there is provided herein a method for separation of bispecific antibodies, comprising the steps of:
a) contacting a liquid sample comprising kappa light chain-containing proteins with a separation matrix,
b) washing said separation matrix with one or a combination of several washing liquids,
c) eluting the kappa light chain-containing protein from the separation matrix with an elution liquid and at a decreasing pH and
d) cleaning the separation matrix with a cleaning liquid,
wherein the IgG capacity of the separation matrix after 24 h incubation in 0.1M NaOH at 22+/−2° C. is at least 80%, or at least 85%, or at least 90%, or at least 95% of the IgG capacity before the incubation.

According to one embodiment, the separation matrix as disclosed above is used in said method.

The method above may also be used for separation of tri-specific antibodies.

The pH may be decreased by using a pH gradient. Alternatively, the pH may be decreased in a stepwise manner, similar to a gradient, using buffer solutions of different pH. The pH range during elution may be from about 5.5 to about 2, such as from about 5 to about 2, from about 4.5 to about 2, or from about 4 to about 2. Apart from the pH, the elution liquid in the separation method is as disclosed for the isolating method above.

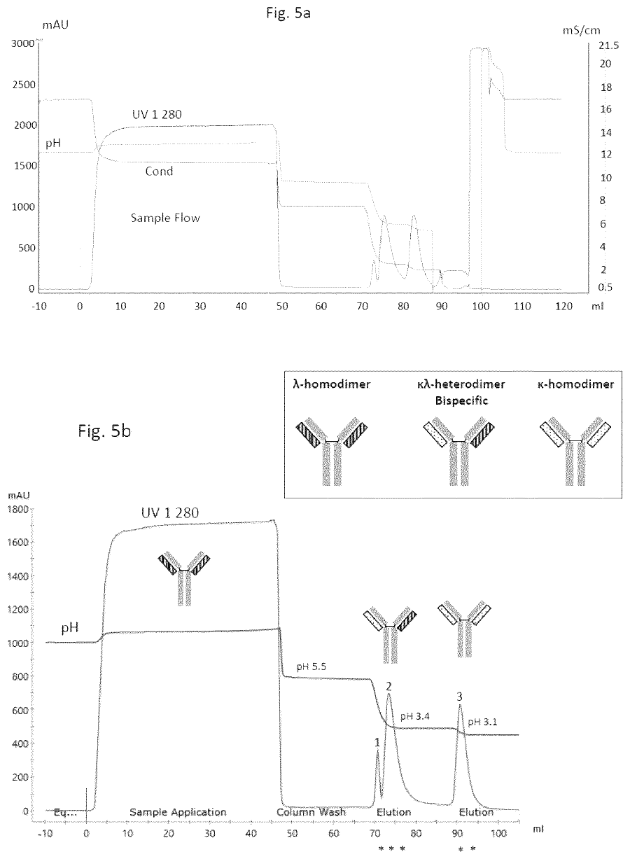

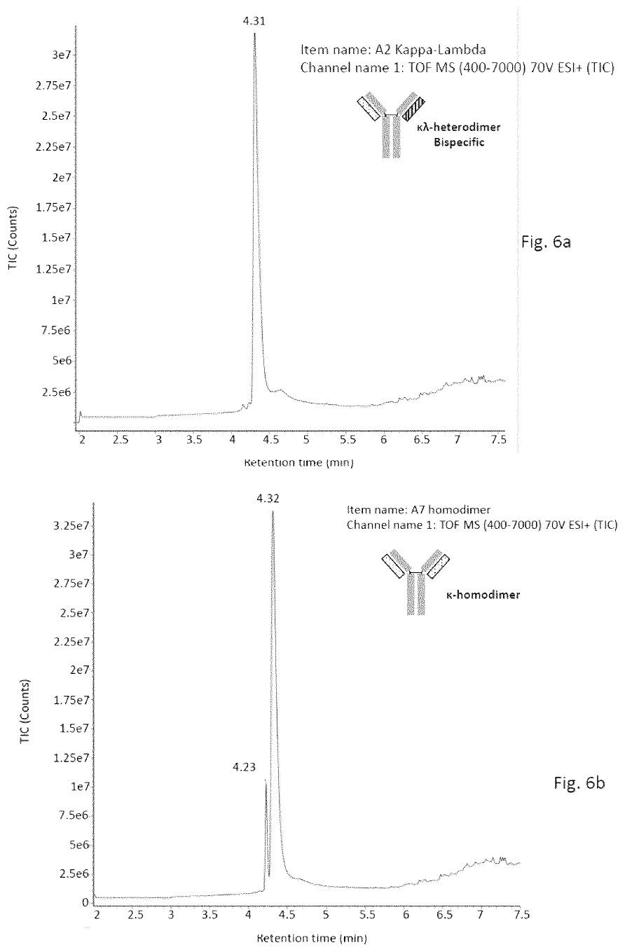

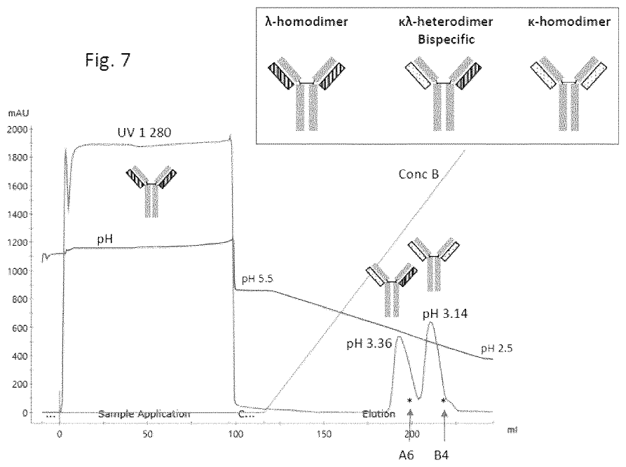

This method enables to separate bi-specific antibodies from monospecific antibodies, or mismatched antibodies, based on the presence of a kappa light chain. A monoclonal antibody has two identical kappa light chain. A bispecific antibody can be designed to comprise two different kappa light chain. FIG. 5-7 illustrates results of the method disclosed above, where FIGS. 5 and 6 shows the result of step-by-step decrease of the pH, and FIG. 7 shows the result of a pH gradient for decreasing the pH.

Any antibody not comprising a kappa light chain, such as two lambda light chains, will not bind the separation matrix and consequently be present in the effluent flow during step a) or be washed away during step b). Any antibodies that have at least one kappa light chain will bind to the separation matrix. Upon elution, antibodies that have only one kappa light chain will elute prior to antibodies that have two kappa light chains. This is illustrated by schematic imagers of the respective antibodies and where they elute, or are present in the effluent, in FIGS. 5b and 7.

By using the separation matrix as disclosed above, which binds to subclass 1, 3 or 4 kappa light chains, it is also possible to separate antibodies or antibody fragments comprising one or two kappa light chains of subclass 2 from antibodies or antibody fragments comprising one or two kappa light chains of subclass 1, 3 or 4. By the same principle as above, an antibody comprising two kappa light chains of subclass 2, or one kappa light chain of subclass 2 and a lambda light chain, will not bind the separation matrix and consequently be present in the effluent flow during step a) or be washed away during step b). An antibody with one kappa light chain of subclass 2 and one kappa light chain of any of subclasses 1, 3 or 4 will elute prior to an antibody with two kappa light chains of any of subclasses 1, 3 and 4.

By decreasing the pH in the elution step in a stepwise manner, as shown in FIG. 5b, three distinctive peaks are formed. MS analysis of the eluates showed that the eluate from peak 1 comprised lambda light chain dimers, peak 2 comprised dimers comprising a kappa light chain and a lambda light chain, and peak 3 comprised kappa light chain dimers, See FIG. 7. By decreasing the pH in a pH gradient, only two peaks were observed. MS analysis of the eluates (data not shown) showed that the first peak comprised dimers comprising a kappa light chain and a lambda light chain, and the second peak comprised kappa light chain dimers.

The methods above may also comprise steps of, before step a), providing an affinity separation matrix as described above and providing a liquid sample solution comprising a kappa light chain-containing protein and at least one other substance as a liquid sample. The method may further comprise a step of equilibrating the separation matrix with an equilibration buffer, before adding the liquid sample.

The washing liquid is normally a buffer that is similar or identical to the equilibration buffer. Normally more than one washing liquid is used. For instance, a first washing liquid may have a high salt content and neutral pH, followed by a second washing liquid with no salt and a lower pH. Using a high salt content in the washing liquid will improve the removal of impurities.

The liquid sample comprising a kappa light chain-containing protein and at least one other substance may comprise host cell proteins (HCP), such as Chinese hamster ovary (CHO) cell, E. coli or yeast cell proteins. Contents of CHO cell and E. coli proteins can conveniently be determined by immunoassays directed towards these proteins, e.g. the CHO HCP or E. coli HCP ELISA kits from Cygnus Technologies. The host cell proteins or CHO cell/E. coli/yeast proteins may be desorbed during step b).

The methods above may also comprise, after step c), a step of recovering the eluate and optionally subjecting the eluate to further separation steps, e.g. by anion or cation exchange chromatography, multimodal chromatography and/or hydrophobic interaction chromatography. Suitable compositions of the liquid sample, the washing liquid and the elution liquid, as well as the general conditions for performing the separation are well known in the art of affinity chromatography and in particular in the art of Protein L chromatography.

The cleaning liquid may preferably be alkaline, such as with a pH of 12-14. Such solutions provide efficient cleaning of the matrix, in particular at the upper end of the interval. The cleaning liquid may comprise 0.01-1.0 M NaOH or KOH, such as 0.05-1.0, or 0.05-0.5, or 0.05-0.3, or 0.05-0.1 M NaOH or KOH. The high stability of the separation matrix of the invention enables the use of such comparatively strong alkaline solutions.

The IgG capacity of the separation matrix according to the above, after 24 h incubation in 0.1M NaOH at 22+/−2° C., is at least 80%, or at least 85%, or at least 90%, or at least 95% of the IgG capacity before the incubation.

The IgG capacity of the separation matrix, after cleaning with 130 cycles in 0.3M NaOH, 15 min contact time per cycle, at 22+/−2° C., is at least about 90% of the IgG capacity before the incubation. In an alternative language, the IgG capacity of the separation matrix, after 32 h incubation in 0.3 NaOH, at 22+/−2° C., is at least about 90% of the IgG capacity before the incubation.

The IgG capacity of the separation matrix after cleaning with 50 cycles in 0.5M NaOH, 15 min contact time per cycle, at 22+/−2° C., is at least 95%, or at least 93% of the IgG capacity before the incubation. In an alternative language, the IgG capacity of the separation matrix, after 12 h incubation in 0.5 NaOH, at 22+/−2° C., is at least 95%, or at least 93% of the IgG capacity before the incubation.

Thus, the separation matrix of the present disclosure can withstand cleaning with an alkali solution at a higher concentration than what is commonly used for Protein L ligand-based separation matrices.

The cleaning of the separation matrix is preferably a CIP process (Cleaning-In-Place), which is a process well-known to the skilled person.

Steps a)-d) may be repeated at least 10 times, such as at least 50 times or 50-200 times. This is important for the process economy in that the matrix can be re-used many times. This is shown in FIGS. 1c and 3, where the separation matrix according to the above maintains a high Qb10% also after 100+ cycles of CIP.

The high capacity for the present separation matrix provides productivity advantages in biomanufacturing. Improved alkali stability leads to a more robust process and long lifetime of the resin which adds to the overall process economy. Hence, the present separation matrix leads to a significantly improved process economy for the separation step. It also adds value with respect to critical process parameters compared to other commercially available product.

### EXPERIMENTAL SECTION

### Ligand Characterization

### Example 1—Affinity Test for Ligands—Biacore

The protein L variants were immobilized using amine coupling onto CM5 Chips (Cytiva™, Sweden) (see Biacore Sensor Surface Handbook, https://cdn.cytivalifesciences.com/dmm3bwsv3/AssetStream.aspx?mediaformatid=10061&destinatio nid=10016&assetid=16475).

EDC/NHS activated CM5 surface in 420 s flow rate 10 μl/min over both flow cells, followed by a wash of the system (not sensor surface) with ethanolamine. The variants in immobilization buffer were injected over flow cell 2 and immobilized on activated CM5 surface. Ethanolamine was injected over both flow cells, 420s flow rate 10 μl/min to deactivate surface.

### Solutions:

EDC 0.4 M 1-ethyl-3-(3-dimethylaminopropyl)-carbodiimide in water
NHS 0.1 M N-hydroxysuccinimide in water
Ethanolamine 1 M ethanolamine-HCl pH 8.5
Ligand Typically 10 to 50 μg/mL in immobilization buffer

| Injection procedure: | Flow rate | Contact time |
|---|---|---|
| 1. EDC/NHS (activate the surface) | 10 μL/min | 7 min |
| 2. Ethanolamine (does not pass over the | WASH | — |
| surface) | | |
| 3. Ligand | 10 μL/min | 7 min |
| 4. Ethanolamine (deactivate excess reactive | 10 μL/min | 7 min |
| groups) | | |

Thereafter the protein L variants were assessed for their binding and apparent affinity towards IgG (Gammanorm). The following assay conditions were used:

Analyte: Gammanorm diluted in HBS-EP+ (General purpose running buffer; 0.1 M HEPES, 1.5 M NaCl, 0.03 M EDTA and 0.5% v/v Surfactant P20; from Cytiva™)

Analyte concentrations: 0.026, 0.053, 0.106, 0.2125, 0.425, 0.85, 1.7, 3.4 μM

Regeneration: 10 mM glycine pH 1.5

Assay: 10 min association à 10 min dissociation à 2×regeneration; flow: 10 μl/min

The evaluation was performed in Biacore Insight Evaluation Software, where kinetic and affinity fitting to sensorgrams (not shown) were performed. Kp for steady state affinity and 1:1 kinetics were retrieved from the software.

TABLE 1
Protein L variants with specified amino acids at position
10, 45 and 60, with the KD values for steady state interaction
and 1:1 kinetics. The table is sorted on the lowest steady
state affinity KD first. pAM114 is wt B3 domain.

| | | | | | Steady state | |
|---|---|---|---|---|---|---|
| | | Pos | Pos | Pos | affinity | Kinetic |
| Construct | Domain | 10 | 45 | 60 | KD (nM) | 1:1 (nM) |
| pAM247* | C4-1 | H | H | Q | 38 | 9 |
| pAM254* | B5b-2 | H | H | Y | 41 | 7 |
| pAM261* | C4-2 | H | H | Y | 43 | 12 |
| pAM258* | C2b-2 | H | H | Y | 45 | 17 |
| pAM114* | B3 | N | N | N | 46 | 11 |
| pAM262* | D1-2 | H | H | Y | 48 | 11 |
| pAM246* | C3b-1 | H | H | Q | 49 | 17 |
| pAM244* | C2b-1 | H | H | Q | 50 | 18 |
| pAM260* | C3b-2 | H | H | Y | 51 | 18 |
| pAM253* | B4-2 | H | H | Y | 54 | 14 |
| pAM251* | B2-2 | H | H | Y | 55 | 20 |
| pAM248* | D1-1 | H | H | Q | 56 | 16 |
| pAM239* | B4-1 | H | H | Q | 60 | 17 |
| pAM252 | B3-2 | H | H | Y | 69 | 16 |
| pAM237* | B2-1 | H | H | Q | 72 | 27 |
| pAM240* | B5b-1 | H | H | Q | 79 | 15 |
| pAM249 | B1-2 | H | H | Y | 79 | 29 |
| pAM238 | B3-1 | H | H | Q | 82 | 25 |
| pAM243 | C2-1 | H | H | Q | 83 | 39 |
| pAM257 | C2-2 | H | H | Y | 98 | 47 |
| pAM250 | B1b-2 | H | H | Y | 99 | 40 |
| pAM235 | B1-1 | H | H | Q | 101 | 42 |
| pAM242 | C1b-1 | H | H | Q | 116 | 49 |
| pAM259 | C3-2 | H | H | Y | 120 | 55 |
| pAM236 | B1b-1 | H | H | Q | 130 | 61 |
| pAM245 | C3-1 | H | H | Q | 134 | 65 |
| pAM256 | C1b-2 | H | H | Y | 134 | 59 |
| pAM241* | C1-1 | H | H | Q | 187 | 87 |
| pAM255* | C1-2 | H | H | Y | 324 | 146 |

### Example 2—Alkali Stability for Ligands—Biacore

The immobilised protein L ligands were assessed for NaOH stability. The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva™)
Analyte concentration: 3.4 μM
Regeneration: 10 mM glycine pH 1.5
NaOH concentration: 100 mM
Assay: 10 min analyte association a 1 min analyte dissociation à 10 min NaOH injection à 1 min NaOH dissociation à 1 min wait à 2×regeneration à 1 min wait->repeat 100 cycles. Flow: 10 μl/min

The evaluation was performed in Biacore Insight Evaluation software, where the responses at the report point “Binding late” were collected. Cycle 1 (start-up) and cycle 2 (first cycle of analysis) were excluded from evaluation. The responses in the first evaluation cycle (cycle 3) were set to 100% and the responses in the following 99 cycles were set to a percentage of the responses from the first cycle.

TABLE 2
Protein L variants with specified amino acids with
the percent binding capacity after 100 cycles of
0.1M NaOH. The table is sorted on the highest capacity
after 100 cycles first. pAM114 is wt B3 domain.

| | | | | | % Capacity after |
|---|---|---|---|---|---|
| Construct | Domain | Pos 10 | Pos 45 | Pos 60 | 100 cycles |
| pAM260* | C3b-2 | H | H | Y | 70 |
| pAM252* | B3-2 | H | H | Y | 68 |
| pAM246* | C3b-1 | H | H | Q | 66 |
| pAM258* | C2b-2 | H | H | Y | 63 |
| pAM251* | B2-2 | H | H | Y | 62 |
| pAM244* | C2b-1 | H | H | Q | 61 |
| pAM253* | B4-2 | H | H | Y | 61 |
| pAM261* | C4-2 | H | H | Y | 61 |
| pAM262* | D1-2 | H | H | Y | 61 |
| pAM247* | C4-1 | H | H | Q | 59 |
| pAM238* | B3-1 | H | H | Q | 57 |
| pAM248* | D1-1 | H | H | Q | 57 |
| pAM237 | B2-1 | H | H | Q | 51 |
| pAM239 | B4-1 | H | H | Q | 51 |
| pAM256 | C1b-2 | H | H | Y | 51 |
| pAM250 | B1b-2 | H | H | Y | 46 |
| pAM249 | B1-2 | H | H | Y | 45 |
| pAM254 | B5b-2 | H | H | Y | 44 |
| pAM242 | C1b-1 | H | H | Q | 43 |
| pAM255 | C1-2 | H | H | Y | 41 |
| pAM257 | C2-2 | H | H | Y | 41 |
| pAM259 | C3-2 | H | H | Y | 41 |
| pAM236 | B1b-1 | H | H | Q | 35 |
| pAM243* | C2-1 | H | H | Q | 35 |
| pAM245* | C3-1 | H | H | Q | 35 |
| pAM241* | C1-1 | H | H | Q | 34 |
| pAM235 | B1-1 | H | H | Q | 32 |
| pAM114 | B3 | N | N | N | 29 |
| pAM240* | B5b-1 | H | H | Q | 22 |

The results show that all variants apart from pAM240 have a better NaOH stability compared to pAM114 (wt B2 domain). The HHY variant seems to have a slightly better NaOH stability compared to HHQ. However, in general both variants are shown to perform well. The NaOH stability is mainly domain dependent, where B5, C1, C2 and C3 show the lowest alkali stability. C3b, B3, C2b, B2, B4, C4 and D1 all show a very good alkali stability, for both of the variants HHY and HHQ.

From the above disclosed experiments, it is clear that the mutations N10H, N45H, and N60Y or N60Q have a positive effect on the NaOH stability on Protein L, and that the affinity is maintained. It is notable that the effect is shown on a broader range of Protein L domains. The effect is shown, in particular, for the B2 domain, the B3 domain, the B4 domain, the C2b domain, the C3b domain, the C4 domain and the D1 domain.

### Separation Matrix

For the prototypes tested, the ligand B2-1 (pAM237), corresponding to SEQ ID NO:3, was used. Pentamers of SEQ ID NO: 3 were expressed and purified by conventional means known to the skilled person

### Example 3—Coupling & Packing

The purified pentamer of SEQ ID NO:3 was immobilized on agarose beads as a base matrix according to the exemplary method below.

### Activation

The base matrix used was rigid cross-linked agarose beads with the indicated volume-weighted median diameter, prepared according to the methods of U.S. Pat. No. 6,602,990 and with the indicated pore size corresponding to an inverse gel filtration chromatography Kav value of 0.70 for dextran of Mw 110 kDa, according to the methods described in Gel Filtration Principles and Methods, Pharmacia LKB Biotechnology 1991, pp 6-13.

25 mL (g) of drained base matrix, 10.0 ml distilled water and 2.02 g NaOH(s) was mixed in a 100 ml flask with mechanical stirring for 10 min at 25° C. 4.0 ml of epichlorohydrin was added and the reaction progressed for 2 hours. The activated gel was washed with 10 gel sediment volumes (GV) of water.

### Coupling

To a protein water solution, sodium carbonate (0.01 M), sodium bicarbonate (0.1 M), sodium chloride (0.15 M) and EDTA disodium salt (1 mmol) were added and when all had dissolved, Dithiothreitol (DTT, 0.1 M) was added. The pH was adjusted to above pH 8.0. The reaction mixture was placed on a shaking table (23° C., 500 rpm) and left to reduce for 2 hours.

PD10 prepacked gel filtration columns (Cytiva™) were used to desalt the protein. The columns were equilibrated with desalting solution (0.15 NaCl, 1 mM EDTA) prior to loading the protein (max 2.5 mL). The eluted fractions were collected and combined.

The protein concentration of the desalted solution was determined by UV absorbance at 276 nm with a protein extinction coefficient of 1.0.

The activated gel was washed with 5 GV 0.1M Trisaminomethane (Tris) buffer pH 8.4. 15 mL gel, 20 mg ligand/mL gel ((11.7 mL), 3.3 mL Tris buffer and 7.0 g sodium sulfate were mixed in a 50 mL flask and stirred at 33° C. for 4 h.

After immobilization the gel was washed with 3×1 GV distilled water. The gel and 1 GV (0.1 M phosphate/1 mM EDTA/7.5% Thioglycerol pH 8.5) were mixed and the flasks were left stirring at room temperature for 15-20 h. The gels were then washed 3 times alternately with 3×1GV 0.5 M HAc and 3×1GV 0.1 M TRIS/0.15 M NaCl pH 8.5 and then with 10×1GV ml distilled water. The gel was conditioned in 20% EtOH in a 50% slurry.

### Determination of Ligand Density Through Amino Acid Analysis, AAA

All synthesized prototypes were dried, and the dry weight determined. The skilled person is aware of generally known methods for performing such a procedure. Thereafter, the prototypes were sent dried for amino acid analysis. With the corresponding dry weights and an excel calculation sheet containing information of the protein size and all data on the primary amino sequence of the protein the ligand densities could be derived in mg ligand/mL resin.

### Packing of Separation Matrix Prototype

2 ml of resin was packed in TRI CORN™ 5/100 columns (Cytiva™).

### Example 4—Dynamic Binding Capacity (DBC)

### Preparation of Trastuzumab Sample

Herceptin (Trastuzumab) solution was diluted to 2 mg/mL. The concentration of the sample solution was determined by triplicate measurements of the absorbance spectrophotometrically (QS High Precision cell, Hellma Analytics). at 280 nm using 1.48 mL/mg*cm as absorptivity coefficient. The Trastuzumab concentration was verified by inline UV-measurement at 280 nm of a 2 mm UV-cell in Unicorn 7.5.

### Determination of the 100% Absorbance Signal

PBS buffer was run through the bypass position until a stable baseline is reached. Auto zero, and Trastuzumab solution was applied through bypass to obtain a stable 100% signal, 6 min RT. The absorbance value was noted. PBS buffer was run through the bypass position until a stable baseline is reached.

### DBC Measurements

Adsorption/equilibration buffer: Phosphate buffer 20 mM+0.15 M NaCl, pH 7.2
Elution buffer: Citrate buffer 50 mM, pH 2.5
CIP: 0.1 M NaOH

The Trastuzumab-sample was loaded to 2 ml columns via t a S-pump (sample pump) on an ÄKTA™ pure 25 M at desired flow rate (depending on residence time) until the UV signal of approx. 20%-of-maximum was reached. The column was then washed with adsorption buffer at flow rate 1 mL/min. The protein was eluted with elution buffer at flow rate 0.8 mL/min.

The column was cleaned with a CIP protocol with 15 Minutes contact time, 0.1 M NaOH at a flow rate of 0.2 mL/min during 3 CV followed by re-equilibrated with adsorption buffer. Columns were cleaned manually with 20% EtOH.

The breakthrough capacity was calculated using Exctensions-DBC Calculations-Analyze, which is an Evaluation tool in Unicorn software used with the ÄKTA™ pure 25 M.

For calculation of breakthrough capacity at 10% (Qb10), equation below was used. That is i.e. the amount of Trastuzumab that is loaded onto the column until the concentration of Trastuzumab in the column effluent is 10% of the Trastuzumab concentration in the liquid sample.

$${q}_{{}_{10 \%}} = \frac{{C}_{0}}{{V}_{C}} [ {V}_{app} - {V}_{sys} - {\int }_{{V}_{sys}}^{{V}_{app}} \frac{A ( V ) - {A}_{sub}}{{A}_{100 \%} - {A}_{sub}} * dv ]$$

A_{100%}=100% UV signal
A_{sub}=absorbance contribution from non-binding mAb
A(V)=absorbance at a given applied volume
V_{C}=column volume
Vₐₚₚ=volume applied until 10% breakthrough
V_{sys}=system dead volume
C₀=liquid sample concentration

Correspondingly, the breakthrough capacity at 80% (Qb80) is i.e. the amount of Trastuzumab that is loaded onto the column until the concentration of Trastuzumab in the column effluent is 80% of the Trastuzumab concentration in the liquid sample.

TABLE 3
DBC data for prototypes N1-N26 and P24, including
main factors DW, d50v and Ligand density.

| | Base matrix | Base matrix | AAA | Qb 10 | Qb 80 |
|---|---|---|---|---|---|
| Sample | DW mg/mL | d50v μm | mg/mL | mg/mL | mg/mL |
| N1 | 63.4 | 57.7 | 11.3 | 72.9 | 82.3 |
| N2 | 71.8 | 59.8 | 10.7 | 71.1 | 82.8 |
| N3 | 62.9 | 66.0 | 11.3 | 68.5 | 80.4 |
| N4 | 69.8 | 67.0 | 11.3 | 67.6 | 80.4 |
| N5 | 63.4 | 57.7 | 16.1 | 74.7 | 90.9 |
| N6 | 71.8 | 59.8 | 17.1 | 65.7 | 89.4 |
| N7 | 62.9 | 66.0 | 15.0 | 70.3 | 87.7 |
| N8 | 69.8 | 67.0 | 15.1 | 64.4 | 89.1 |
| N9 | 63.4 | 57.7 | 10.1 | 66.6 | 73.9 |
| N10 | 71.8 | 59.8 | 12.6 | 72.1 | 86.5 |
| N11 | 62.9 | 66.0 | 9.5 | 63.2 | 71.4 |
| N12 | 69.8 | 67.0 | 10.1 | 62.3 | 72.4 |
| N13 | 63.4 | 57.7 | 14.7 | 73.3 | 85.4 |
| N14 | 71.8 | 59.8 | 17.1 | 67.4 | 90.7 |
| N15 | 62.9 | 66.0 | 13.1 | 67.4 | 81.5 |
| N16 | 69.8 | 67.0 | 14.0 | 63.1 | 81.6 |
| N17 | 66.4 | 63.3 | 13.6 | 70.6 | 86.5 |
| N18 | 66.4 | 63.3 | 13.2 | 69.8 | 85.4 |
| N19 | 66.4 | 63.3 | 14.8 | 69.8 | 85.4 |
| N20 | 66.4 | 63.3 | 13.5 | 71.9 | 87.1 |
| N21 | 66.4 | 63.3 | 11.0 | 69.6 | 79.6 |
| N22 | 66.4 | 63.3 | 15.3 | 69.0 | 85.6 |
| N23 | 63.4 | 57.7 | 14.2 | 73.1 | N/A |
| N24 | 62.9 | 66.0 | 13.5 | 69.0 | N/A |
| N25 | 63.4 | 57.7 | 16.7 | 75.6 | N/A |
| N26 | 62.9 | 66.0 | 16.6 | 68.5 | N/A |
| P24 | 66.5 | 62.2 | 17.5 | 62.1 | 82.7 |

A selection of the ligand densities was further tested for alkali stability.

TABLE 4
Remaining capacity for the indicated
ligand densities with Trastuzumab

| | Remaining capacity after 112 |
|---|---|
| Ligand density | CIP cycles with 0.1M NaOH |
| 15.5 | 96.2 |
| 11.9 | 85.2 |
| 17.8 | 100.5 |
| 19.0 | 99.0 |
| 14.6 | 96.4 |
| 16.9 | 99.8 |
| 16.1 | 93.4 |
| 10.4 | 82.5 |
| 20.4 | 102.6 |

The results from Table 4 are plotted in FIG. 1a, where a clear correlation between ligand density and alkali stability can be observed.

Additionally, a selection of ligand densities was tested for DBC at 10% breakthrough with a Fab-fragment at 6 min residence time.

The Fab-fragment was produced from Trastuzumab by papain cleavage. The Trastuzumab solution was adjusted to pH 7.4 by addition of 0.5 M Sodium phosphate and then diluted 1+1 in digestion buffer (25 mM Na-phosphate, 1 mM EDTA, 5 mM mercapto-ethanol, pH 7.5). Final volume was approx. 100 mL Papain crystals were added to the solution. The solution was incubated at 37° C. over-night. Thereafter, Antipain (papain inhibitor) was added to the digested Trastuzumab. The solution was left in room temperature for 30 min prior to application onto a Capto™ L HiScale 26 column to remove Fc-containing molecules (Fc or partially digested Trastuzumab, collected in flow through). Fab was collected during elution.

TABLE 5
Remaining capacity for the indicated
ligand densities with a Fab-fragment

| Ligand density | DBC Qb10% 6 min RT |
|---|---|
| 10.6 | 56.8 |
| 15.0 | 66.7 |
| 13.0 | 62.4 |
| 16.1 | 67.9 |
| 11.0 | 55.5 |
| 15.3 | 66.4 |

The results of Table 5 are plotted in FIG. 1b, where again a clear correlation between ligand density and DBC can be observed.

FIG. 2 illustrates DBC in relation to ligand density, Kd and Dw. IT is clear that the herein disclosed range for KD, d50V and ligand density for the alkali stabilized Protein L domains disclosed herein, leads to an advantageous DBC.

Next, a selection of ligand densities were tested specifically for alkali stability.

TABLE 6
Test of 5 prototypes with the indicated
ligand densities and the relative DBC.

| | | Start DBC QB10 | Relative remaining Qb10 |
|---|---|---|---|
| | Ligand density | Trastuzumab, RT | after 112 CIP cycles with |
| Sample | (mg/mL) | 4 min (mg/mL) | 0.1M NaOH (%) |
| N8 | 19.0 | 58.6 | 99.0 |
| N11 | 14.6 | 61.9 | 96.4 |
| N17 | 16.07 | 61.9 | 93.4 |
| N2 | 11.9 | 66.2 | 85.2 |
| C22 | 10.4 | 58.8 | 82.5 |

As can be seen in FIG. 1c, all of the prototypes show a Qb10% of above 80% after 112 CIP cycles. In particular, the prototypes with ligand densities that fall within the range disclosed for the present invention show a Qb10% of above 90%. The least performing prototypes have a ligand density of below 12 mg/ml.

### Example 5—Alkali Stability

DBC for three different ligand densities (Base matrix: KD 0.75, Dw 66.5 mg/ml and d50v 62.3 μm) were tested and compared with two commercially available products during a CIP process. The antibody used in the test was Trastuzumab.

Trastuzumab was diluted to 2 mg/mL. The concentration can be determined according to any well-known method within the technical field.

The indicated prototypes were included in an accelerated alkaline stability study to investigate how the stability is dependent on ligand density. Capto™ L was included for reference. Data of DBC Qb_{10%} at 4 min residence time with Trastuzumab, measured between the incubations in 100 mM NaOH, are presented in Table 5.

A strip step was performed with 50 mM Citrate pH 2.3 followed by equilibration (20 mM Sodium Phosphate+150 mM NaCl pH 7.2) prior to the incubation in 100 mM NaOH. The incubation is performed for 4 hours corresponding to 16 cycles of CIP with 15 min contact time. DBC Qb_{10%} with Trastuzumab at 4 min residence time was measured between the incubation runs. The dynamic binding capacity is measured with Trastuzumab and the relative remaining capacity, to the initial DBC, is calculated between the incubations corresponding to a total of 100 cycles with CIP.

TABLE 7
Tested ligand densities vs Capto ™ L, DBC at different cycles.

| | Prototype | Prototype | |
|---|---|---|---|
| CIP cycle | 14.2 mg/mL | 17.7 mg/mL | Capto ™ L* |
| 1 | 58 | 39 | 25 |
| 18 | 58 | 39 | 23 |
| 35 | 56 | 39 | 21 |
| 52 | 56 | 40 | 19 |
| 65 | 55 | 42 | 18 |
| 82 | 51 | 40 | 15 |
| 100 | 49 | 42 | 14 |
| Relative remaining DBC after | 85 | 109 | 55 |
| 100 CIP cycles (%) | | | |

*Commercially available from Cytiva ™

The results above are shown in FIG. 3a. The same results re-calculated as relative DBC are shown in FIG. 3b. The DBC Qb10% values at start show that the Prototype 17.7 mg/ml presents a lower capacity compared to the other prototype of 14.2 mg/ml. However, the DBC Qb10% for the Prototype 17.7 mg/ml increases with number of CIP-cycles i.e., incubation time in NaOH, indicating a better alkaline stability compared to the other prototype of 14.2 mg/ml (FIG. 3a).

To compare the separation matrices with regard to alkaline stability, the relative remaining DBC Qb10% to the start DBC Qb10% value was calculated over the number of CIP cycles. After 100 cycles, i.e., totally 25 hours of incubation in 100 mM NaOH, all prototypes included in this study achieve more than 80% remaining capacity.

Thus, it is apparent that both prototype s tested, with a ligand density of 14.2 mg/ml and 17.7 mg/ml, have a very good DBC after 100 cycles, showing a higher DBC than Capto™ L.

Further it was tested if a separation matrix according to the present disclosure can endure cleaning under alkali concentration that are higher than what is conventionally used for Protein L separation matrices. Tricorn 5/50 columns were packed with a prototype resin comprising 17.7 mg/ml ligand, as disclosed above.

### 300 mM NaOH

The columns was subjected to 15 min CIP with 300 mM NaOH/cycle, using Trastuzumab. DBC Qb10% was determined for 3 min residence time. The experiment was performed with an ÄKTA™ pure 25 M.

Equilibration: 20 mM Na-Phosphate 150 mM NaCl pH 7.2, 1 ml/min, 5 CV

Sample load: via sample pump, 0.333 ml/min (3 min RT) to 20% of abs max)

Wash with 0 mM Na-Phosphate 150 mM NaCl pH 7.2, 1 ml/min, 7 CV

Elution: 50 mM Na-Citrate pH 2.5, 1 ml/min, 5 CV

Wash: with Milli-Q water, 1 ml/min, 5 CV

CIP: with 300 mM NaOH, 0.2 ml/min, 3 CV (15 min contact time)

Re-equilibration: 20 mM Na-Phosphate 150 mM NaCl pH 7.2, 1 ml/min, 5 CV

TABLE 8
Results for 15 min CIP, 300 mM NaOH/Cycle,
3 minutes RT with Trastuzumab.

| | | Relative remaining capacity |
|---|---|---|
| CIP cycles | QB10 | % |
| 0 | 52.3 | 100 |
| 10 | 51.9 | 99 |
| 20 | 52.6 | 100 |
| 30 | 53.7 | 103 |
| 40 | 54.7 | 105 |
| 50 | 56.6 | 108 |
| 60 | 56.9 | 109 |
| 70 | 57.5 | 110 |
| 80 | 57.1 | 109 |
| 90 | 56.0 | 107 |
| 100 | 54.1 | 103 |
| 110 | 49.9 | 96 |
| 120 | 50.0 | 96 |
| 130 | 46.5 | 89 |

### 500 mM NaOH

The column was subjected to performed with 15 min CIP with 500 mM NaOH/cycle with Trastuzumab. DBC Qb10% was determined for 4 min residence time. The experiment was performed with an ÄKTA™ pure 25 M.

Equilibration: 20 mM Na-Phosphate 150 mM NaCl pH 7.2, 1 ml/min, 5 CV

Sample load: via sample pump, 0.25 ml/min (4 min RT) to 20% of abs max)

Wash with 0 mM Na-Phosphate 150 mM NaCl pH 7.2, 1 ml/min, 7 CV

Elution: 50 mM Na-Citrate pH 2.5, 1 ml/min, 5 CV

Wash: with Milli-Q water, 1 ml/min, 5 CV

CIP: with 500 mM NaOH, 0.2 ml/min, 3 CV (15 min contact time)

Re-equilibration: 20 mM Na-Phosphate 150 mM NaCl pH 7.2, 1 ml/min, 5 CV.

TABLE 9
Result for 15 min CIP, 500 mM NaOH/Cycle,
4 minutes RT with Trastuzumab.

| | | Relative remaining capacity |
|---|---|---|
| CIP cycles | QB10 | % |
| 0 | 60.4 | 100 |
| 10 | 61.7 | 102 |
| 20 | 63.0 | 104 |
| 30* | — | — |
| 40* | — | — |
| 50 | 56 | 93 |
| 60 | 50 | 83 |
| 70 | 44 | 72 |

*Data not shown due to a faulty residence time.

As can be seen in FIG. 3c, where the relative remaining capacities for 0.1M NaOH, 0.3M NaOH and 0.5M NaOH are plotted, the separation matrix of the present disclosure has an excellent alkali stability also for the higher alkali concentrations. For 0.1M NaOH, the separation matrix has a 90% remaining capacity after 200 cycles. For 0.3M NaOH, the separation matrix has an approximately 90% remaining capacity after 130 cycles, and for 0.5M NaOH the separation matrix has an approximately 90% remaining capacity after 50 cycles.

### Example 6—Comparative Test-DBC

Next, DBC Qb_{10%} with Adalimumab, Fab, dAb and Trastuzumab was measured at residence time 1, 2.4, 4, 6 and 10 min for a prototype (with ligand density about 14.3 mg/ml, KD of about 0.75, Dw of about 66.5 mg/ml and a d50v of about 62.3 μm).

Fab was produced as disclosed above. dAb was produced according to the method described in the article “Recombinant production of a V_{L} single domain antibody in Escherichia coli and analysis of its interaction with Peptostreptococcal protein L” (Protein Expression and Purification, Volume 51, Issue 2, February 2007, Pages 253-259)

The data is presented in Table 10 together with DBC Qb_{10%}.

TABLE 10

DBC Qb_{10%} (mg/mL)

| Target sample | 1 min | 2.4 min | 4 min | 6 min | 10 min |
|---|---|---|---|---|---|
| Adalimumab | 22 | N/A | 57 | 67 | 75 |
| Fab | 48 | 62 | 66 | 69 | 70 |
| dAb | 38 | 40 | 40 | 40 | 41 |
| Trastuzumab | 22 | 44 | 59 | 72 | 82 |

The results show that DBC Qb_{10%} with Fab reach an optimum from 6 min residence time and higher, whereas DBC Qb_{10%} with mAb may possibly increase further with higher residence times. The kinetics for dAb is very fast and DBC Qb_{10%} for the smaller fragment reach an optimum already at 2.4 min residence time for the prototype

Thereafter, the prototype resin was compared to three commercially available Protein L resins. The prototype resin was packed in triplicate Tricorn 5/100 columns and DBC was performed according to the methods described above. DBC Qb10% was determined at the indicated residence times with an antibody or an antibody fragment for the prototype, TOYOPEARL® AF-r Protein L-650F (Tosoh), KanCap™ L (KaneKa) and Capto™ L (Cytiva™). An average of data from triplicate columns are presented below.

TABLE 11
DBC with Trastuzumab at different residence times

| | DBC at Qb_{10%} with Trastuzumab | | | | | |
|---|---|---|---|---|---|---|
| Resin | 1 min | 2.4 min | 4 min | 6 min | 8 min | 10 min |
| Prototype | 22 | 44 | 59 | 72 | 79 | 82 |
| TOYOPEARL* | 27 | 44 | 51 | 54 | 56 | 59 |
| Capto ™ L | 18 | 28 | 32 | 34 | 35 | 36 |
| KanCap ™ L | 17 | 35 | 45 | 50 | 53 | 57 |

*TOYOPEARL ® AF-r Protein L-650F

The results, shown in FIG. 4a, show that for a residence time of 1 min, the prototype performs slightly better than Capto™ L and KanCap™ L while TOYOPEARL® AF-r Protein L-650F present a higher capacity. However, for residence time 2.4 min, the prototype and TOYOPEARL® AF-r Protein L-650F are comparable and for 6, 8 and 10 min residence time, the prototype presents significantly higher DBC Qb_{10%} than TOYOPEARL® AF-r Protein L-650F.

Compared to Capto™ L, the prototype presents a two-fold higher DBC Qb_{10%} for the higher residence times (6-10 min).

DBC was also determined with a Fab-fragment at the indicated residence times on duplicate columns with the indicated resins according to the methods described above.

TABLE 12
DBC Qb10% DBC with Fab-fragment at different residence
times for prototype, TOYOPEARL ® AF-r Protein
L-650F (Tosoh), Capto ™ L (Cytiva ™) and KanCap ™ L (KaneKa).

| Resin | 1 min | 2.4 min | 4 min | 6 min | 10 min |
|---|---|---|---|---|---|
| Capto ™ L | 16.9 | 19.4 | 20.4 | 20.5 | 20.9 |
| KanCap ™ L | 32.0 | 41.5 | 44.0 | 46.0 | 46.8 |
| TOYOPEARL* | 43.7 | 53.2 | 56.2 | 57.7 | 58.1 |
| Prototype | 48.1 | 62.2 | 66.4 | 68.8 | 69.7 |

*TOYOPEARL ® AF-r Protein L-650F

The results show that the prototype has a significantly better DBC Qb_{10%} at all residence times for Fab compared to TOYOPEARL® AF-r Protein L-650F and KanCap™ L, as well as compared to Capto™ L, see FIG. 4b.

Furthermore, DBC was determined with dAb at the indicated residence times on duplicate columns with the indicated resins according to the methods described above.

TABLE 13
DBC Qb10% DBC with dAb at different residence
times for prototype, TOYOPEARL (Tosoh), and
Capto ™ L (Cytiva ™).

| Resin | 2.4 min | 4 min | 6 min | 10 min |
|---|---|---|---|---|
| Capto ™ L | 15.8 | 16.2 | 16.4 | 16.7 |
| TOYOPEARL* | 23.7 | 25.2 | 26.3 | 26.0 |
| Protoype | 37.0 | 37.9 | 38.7 | 40.0 |

*TOYOPEARL ® AF-r Protein L-650F

Again, the results show that the prototype has a significantly better DBC Qb10% at all residence times also for dAb compared to TOYOPEARL® AF-r Protein L-650F, as well as compared to Capto™ L, see FIG. 4c.

### Example 7—Separation of Bi-Specific Antibodies

The ability to separate bi-specific antibodies from mono-specific antibodies, based on the kappa light-chain was tested. The separation matrix as in the previous examples, packed in a Tricorn 5/100 column, was used in this experiment.

The Ab-containing liquid sample (bsAb01) tested in this experiment is a commercially available sample from Thermo Fisher, comprising a) a κ-light chain, Trastuzumab Kappa class 1 Anti-HER2 Light chain (1 and 2); b) a λ-Light chain, Avelumab Lambda class 2 Anti PDL1 Light chain; and c) a FC chain, Anti-HER2 Heavy chain (1 and 2). They are present in the sample in a ratio of 30:30:40 respectively.

### Stepwise Decrease of pH at Elution

TABLE 14
Materials used for stepwise elution.

| Chemicals | Supplier |
|---|---|
| Ethanol | TechniSolv |
| NaCl | VWR |
| 20 mM Sodium Phosphate + 150 mM NaCl pH 7.2 | Merck, Merck, |
| (Adsorption/Equilibration buffer) | VWR |
| 50 mM Citrate pH 5.5 (Wash buffer) | Merck, VWR |
| 50 mM Citrate pH 3.4 (Elution buffer 1) | Merck, VWR |
| 50 mM Citrate pH 3.1 (Elution buffer 2) | Merck, VWR |
| 50 mM Citrate pH 2.3 (Strip buffer) | Merck, VWR |
| bsAb01 | Thermo Fisher |
| 0.1M NaOH (CIP) | Titrisol |

The liquid sample was prepared by thawing in room temperature and filtered through a 0.2 μm filter. The concentration of mAb in the liquid sample was estimated to approximately 0.1 mg/ml based on peak integration.

After column equilibration with equilibration buffer, 50 ml of the liquid sample was applied on to 1 ml column at 0.250 ml/min, followed by 10 CV of the wash buffer.

Step elution was performed by first applying 5 CV of pH 3.4 elution buffer 1 and subsequently 5 CV of pH 3.1 elution buffer 2 at 1.0 ml/min flow. After elution, a 3 CV pH 2.3 Strip step was performed and a 3 CV 0.1 M NaOH CIP step followed. Peak fractionation was used to collect fractions. The result is shown in FIG. 5a. This was repeated with elution volumes changed to 10 CV. The result is shown in FIG. 5b.

As can be seen in FIG. 5a, with the initial 5 CV elution volume run, three distinct peaks could be observed, with some tailing and overlap between the major peaks. An initial small peak, which eluted at pH 4.45, was also observed.

With 10 CV elution volumes baseline separation of the KA-heterodimers and KK-homodimers is achieved, as seen in FIG. 5b.

Fractions from the 10 CV run were analysed by Mass-Spectrometry (MS) in full size and reduced form. These fractions are marked with an * in FIG. 5b.

The non-reduced MS results, from fraction 1 (peak 1 in FIG. 5b), indicated that it resembles a full size mAb (data not shown) When the sample was reduced a number of peaks are resolved corresponding to a triplet of light chain peaks, an FC peak and an unknown peak was observed (data not shown). The light chain observed was of identical masses as observed for λ light chain of the κλ-dimer. The FC was of identical mass as observed for the κλ and κκ dimers. This λλ-dimer elutes very readily, at pH 4.45, and can probably be removed or isolated by increased washing of the resin after sample application or a less steep initial pH gradient.

Peak 2 in FIG. 5b was the Bispecific κλ mAb, see FIG. 6a, with a full size of 147406 Da. When reduced two types of light chains could be observed (data not shown).

Peak 3 in FIG. 5b was the κκ-homodimer, see FIG. 6b, where a small fraction of free κ-light chain was be observed. In the reduced MS data (data not shown), only one type of light chain was observed, which corresponded in size to the of the κ-light chain observed for the κλ-bispecific mAb.

Thus, a stepwise pH separation of bispecific kappa-light chain containing monoclonal antibodies using the separation matrix according to the invention is possible. By simple adjustment of elution volumes at each pH baseline, separation of the bispecific heterodimer and the homodimer can be achieved.

Elution with pH Gradient

TABLE 15
Materials used for gradient elution

| Chemicals | Supplier |
|---|---|
| Ethanol | TechniSolv |
| NaCl | VWR |
| 20 mM Sodium Phosphate + 150 mM NaCl pH 7.2 | Merck, Merck, |
| (Adsorption/Equilibration buffer) | VWR |
| 50 mM Citrate pH 2.5 (Elution buffer) | Merck, VWR |
| 50 mM Citrate pH 5.5 (wash buffer) | Merck, VWR |
| BsAb_mAb01 | Thermo Fisher |
| 0.1M NaOH (CIP) | Titrisol |

Adsorption/equilibration buffer: Phosphate buffer 20 mM+0.15 M NaCl, pH 7.2

Elution buffers: 50 mM Citrate buffer, pH 5.5, 50 mM Citrate buffer, pH 2.5

Strip buffer: 50 mM Citrate, pH 2.3

CIP: 0.1 M NaOH

After equilibration, ˜100 ml of the BsAb_mAb01 sample was loaded to 1 ml columns via the B-pump at desired flow rate of 0.25 ml/min. The column was then washed with wash buffer for 10 CV at flow rate 1 mL/min. The protein was eluted with a pH gradient of 50 mM Citrate pH 5.5-2.5 over 40 CV at a flowrate of 0.25 ml/min.

The column was cleaned with strip using 50 mM Citrate pH 2.3 followed by 0.1 M NaOH at a flow rate of 0.4 mL/min and finally re-equilibrated with 10 CV adsorption buffer.

The elution was collected by peak-fractionation starting at 50 mAu.

The bound fractions eluted with two major peaks, A and B, at pH 3.36 and pH 3.12, see FIG. 7.

Presumably these peaks correlate to the Bispecific κλ-heterodimer which elutes first at pH 3.26, and the κκ-homodimer which elutes later at pH 3.12. The λλ-homodimer does not bind and does not elute during the gradient

Samples from fractions A6 and B4, as indicated in FIG. 7, were also reduced, and then analysed with MS (data not shown). This analysis clearly showed that A6 was comprised of a κ-light chain (23439 Da), a λ-light (22785 Da) chains and an FC chain (25297 Da). B4 was shown to be comprised of only a κ-light chain (23439 Da) and FC chains (25297 Da)

Hence, the above examples show that a separation matrix according to the appended claims has an improved alkali stability as well as DBC at 10% breakthrough for all antibodies or antibody fragments tested, compared to similar commercially available products.

This written description uses examples to disclose the invention, and also to enable any person skilled in the art to practice the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Any patents or patent applications mentioned in the text are hereby incorporated by reference in their entireties, as if they were individually incorporated.

1. A separation matrix comprising at least 12 mg/ml kappa light chain-binding ligands covalently coupled to a porous support, wherein
said kappa light chain-binding ligands comprise, consists essentially of, or consists of multimers of alkali-stabilized Finegoldia magna (formerly Peptostreptococcus magnus) Protein L domains; and
said porous support comprises polymer particles having a Dry solids weight (D_{W}) of 50-200 mg/ml, a volume-weighted median diameter (D50v) of 30-100 μm.

2. The separation matrix according to claim 1, wherein said matrix comprises at least 14 mg/ml kappa light chain-binding ligands, such as at least 14.5 mg/ml, at least 15 mg/ml, at least 15.5 mg/ml, at least 16 mg/ml, at least 16.5 mg/ml, at least 17 mg/ml, at least 17.5 mg/ml, at least 18 mg/ml, at least 18.5 mg/ml, or at least 19 mg/ml kappa light chain-binding ligands.

3. The separation matrix according to claim 1, wherein the porous support has a D_{W} of 50-150 mg/ml, 50-120 mg/ml, 50-100 mg/ml, 50-90 mg/ml, 60-80 mg/ml, or 60-75 mg/ml, such as at least 63 mg/ml, or at least 65 mg/ml, or at least 70 mg/ml.

4. The separation matrix according to claim 1, wherein the porous support has a volume-weighted median diameter (D50v) of 35-90 μm, 40-80 μm, 50-70 μm, 55-70 μm, 55-67 μm, 58-70 μm, or 58-67 μm, such as at least 60 μm, or at least 62 μm.

5. The separation matrix according to claim 4, having a Kd value, measured by inverse size exclusion chromatography with dextran of Mw 110 kDa as a probe molecule, of 0.6-0.95, such as a Kd value of 0.7-0.9, or a Kd value of 0.6-0.8, such as a Kd value of about 0.67, or a Kd value of about 0.72, or a Kd value of about 0.75.

6. The separation matrix according to claim 1, wherein the polymer particles are cross-linked.

7. The separation matrix according to claim 1, wherein at least two of the alkali-stabilized Protein L domains are selected from the group comprising of functional variants of a B1 domain, a B2 domain, a B3 domain, a B4 domain, a B5 domain, a C2 domain, a C3 domain, a C4 domain and a D1 domain of Finegoldia magna (formerly Peptostreptococcus magnus) Protein L, wherein the positions which in an alignment corresponds to positions 10 and 45 in a B2 domain (SEQ ID NO 1) are histidine, and the position which in an alignment corresponds to position 60 in a B2 domain (SEQ ID NO 1) is a tyrosine or a glutamine.

8. The separation matrix according to claim 7, wherein the at least two alkali-stabilized Protein L domains are chosen from the group comprising a B2 domain, a B3 domain, a B4 domain, a C2 domain, a C3 domain, a C4 domain and a D1 domain.

9. The separation matrix according to claim 7, wherein the at least two alkali-stabilized Protein L domains have at least 90%, 95% or 98% sequence identity or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19, wherein the positions which in an alignment corresponds to positions 10 and 45 in SEQ ID NO 1, and the position which in an alignment corresponds to position 60 in SEQ ID NO 1 are not variable.

10. The separation matrix according to claim 1, wherein the separation matrix comprises three, four, five, six, seven, eight or nine alkali-stabilized Protein L domains.

11. The separation matrix according to claim 10, wherein the separation matrix comprises four, five or six alkali-stabilized Protein L domains.

12. The separation matrix according to claim 1, wherein the ligand comprises a coupling element, said coupling element being one or more cysteine residues, one or more lysine residues, or one or more histidine residues at the C-terminal end of the ligand.

13. The separation matrix according to claim 12, wherein the ligand comprises one or more cysteine residues at the C-terminal end of the ligand.

14. The separation matrix according to claim 1, wherein the separation matrix has a 10% breakthrough dynamic binding capacity for IgG of at least 55 mg/ml at 4 min residence time.

15. The separation matrix according to claim 1, wherein the separation matrix has a 10% breakthrough dynamic binding capacity for IgG of at least 70 mg/ml at 6 min residence time.

16. The separation matrix according to claim 1, wherein the separation matrix has a 10% breakthrough dynamic binding capacity for IgG of at least 80 mg/ml at 10 min residence time.

17. The separation matrix according to claim 1, wherein the IgG capacity of the separation matrix after 24 h incubation in 0.1M NaOH at 22+/−2° C. is at least 80%, or at least 85%, or at least 90%, or at least 95% of the IgG capacity before the incubation.

18. The separation matrix according to claim 1, wherein the IgG capacity of the separation matrix after 32 h incubation in 0.3M NaOH at 22+/−2° C. is at least 90% of the IgG capacity before the incubation.

19. The separation matrix according to claim 1, wherein the IgG capacity of the separation matrix after 12 h incubation in 0.5M NaOH at 22+/−2° C. is at least 95%, or at least 93% of the IgG capacity before the incubation.

20. A method of isolating a kappa light chain-containing protein comprising the steps of:
a) contacting a liquid sample comprising a kappa light chain-containing protein with a separation matrix;
b) washing said separation matrix with one or a combination of several washing liquids;
c) eluting the kappa light chain-containing protein from the separation matrix with an elution liquid; and
d) cleaning the separation matrix with a cleaning liquid;
wherein the IgG capacity of the separation matrix after 24 h incubation in 0.1M NaOH at 22+/−2° C. is at least 80%, or at least 85%, or at least 90%, or at least 95% of the IgG capacity before the incubation.

21. A method for separation of bispecific antibodies comprising the steps of:
a) contacting a liquid sample comprising kappa light chain-containing proteins with a separation matrix,
b) washing said separation matrix with one or a combination of several washing liquids,
c) eluting the kappa light chain-containing protein from the separation matrix with an elution liquid and at a decreasing pH
d) cleaning the separation matrix with a cleaning liquid,
wherein the IgG capacity of the separation matrix after 24 h incubation in 0.1M NaOH at 22+/−2° C. is at least 80%, or at least 85%, or at least 90%, or at least 95% of the IgG capacity before the incubation.

22. The method of claim 21, wherein decreasing the pH is performed by using a pH gradient.

23. The method of claim 21, wherein decreasing the pH is performed in a stepwise manner.

24. The method according to claim 21, wherein the decreasing pH is from about 5.5 to about 2, such as from about 5 to about 2, from about 4.5 to about 2, or from about 4 to about 2.

25. The method according to claim 20, wherein the cleaning liquid comprises 0.01-1.0 M NaOH or KOH, such as 0.05-1.0 M or 0.05-0.1 M, or 0.05-0.3M, or 0.05-0.5 NaOH or KOH.

26. The method according to claim 20, wherein the elution liquid comprises at least one anion species selected from the group consisting of acetate, citrate, glycine, succinate, phosphate, and formate.

27. The method according to claim 20, wherein steps a)-d) are repeated at least 10 times, such as at least 50 times or 50-200 times.

28. A method of isolating a kappa light chain-containing protein comprising using the separation matrix of claim 1.

29. A chromatography column comprising the separation matrix of claim 1.

30. A method of isolating a kappa light chain-containing protein comprising using the chromatography column of claim 29.


SEQUENCE LISTING (37 sequences)

SEQ ID NO: 1 - protein, 72 aa, Finegoldia magna
PKEEVTIKANLIYADGKTQTAEFKGTFEEAAAEAYRYADALKKDNGEYTVDVADKGYTLNIKFAGKEKTPEE

SEQ ID NO: 2 - protein, 72 aa, synthetic construct
  REGION 1..72: note = Recombinantly produced
PKEEVTIKAHLIYADGKTQTAEFKGTFEEAAAEAYRYADALKKDHGEYTVDVADKGYTLYIKFAGKEKTPEE

SEQ ID NO: 3 - protein, 72 aa, synthetic construct
  REGION 1..72: note = Recombinantly produced
PKEEVTIKAHLIYADGKTQTAEFKGTFEEAAAEAYRYADALKKDHGEYTVDVADKGYTLQIKFAGKEKTPEE

SEQ ID NO: 4 - protein, 72 aa, synthetic construct
  REGION 1..72: note = Recombinantly produced
PKEEVTIKAHLIYADGKTQTAEFKGTFEEATAEAYRYADLLAKEHGKYTVDVADKGYTLYIKFAGKEKTPEE

SEQ ID NO: 5 - protein, 72 aa, synthetic construct
  REGION 1..72: note = Recombinantly produced
PKEEVTIKAHLIYADGKTQTAEFKGTFEEATAEAYRYADLLAKEHGKYTVDVADKGYTLQIKFAGKEKTPEE

SEQ ID NO: 6 - protein, 72 aa, synthetic construct
  REGION 1..72: note = Recombinantly produced
SEEEVTIKAHLIFANGSTQTAEFKGTFEKATSEAYAYADTLKKDHGEYTVDVADKGYTLYIKFAGKEKTPEE

SEQ ID NO: 7 - protein, 72 aa, synthetic construct
  REGION 1..72: note = Recombinantly produced
SEEEVTIKAHLIFANGSTQTAEFKGTFEKATSEAYAYADTLKKDHGEYTVDVADKGYTLQIKFAGKEKTPEE

SEQ ID NO: 8 - protein, 74 aa, synthetic construct
  REGION 1..74: note = Recombinantly produced
PKEEVTIKAHLIYADGKTQTAEFKGTFAEATAEAYRYADLLAKEHGKYTADLEDGGYTIYIRFAGKKVDEKPEE

SEQ ID NO: 9 - protein, 74 aa, synthetic construct
  REGION 1..74: note = Recombinantly produced
PKEEVTIKAHLIYADGKTQTAEFKGTFAEATAEAYRYADLLAKEHGKYTADLEDGGYTIQIRFAGKKVDEKPEE

SEQ ID NO: 10 - protein, 70 aa, synthetic construct
  REGION 1..70: note = Recombinantly produced
KEQVTIKEHIYFEDGTVQTATFKGTFAEATAEAYRYADLLSKEHGKYTADLEDGGYTIQIRFAGKEEPEE

SEQ ID NO: 11 - protein, 70 aa, synthetic construct; identical to SEQ ID NO: 10
  REGION 1..70: note = Recombinantly produced

SEQ ID NO: 12 - protein, 74 aa, synthetic construct
  REGION 1..74: note = Recombinantly produced
PKEEVTIKVHLIFADGKTQTAEFKGTFEEATAKAYAYADLLAKEHGEYTADLEDGGYTIYIKFAGKETPETPEE

SEQ ID NO: 13 - protein, 74 aa, synthetic construct
  REGION 1..74: note = Recombinantly produced
PKEEVTIKVHLIFADGKTQTAEFKGTFEEATAKAYAYADLLAKEHGEYTADLEDGGYTIQIKFAGKETPETPEE

SEQ ID NO: 14 - protein, 74 aa, synthetic construct
  REGION 1..74: note = Recombinantly produced
PKEEVTIKVHLIFADGKIQTAEFKGTFEEATAKAYAYADLLAKEHGEYTADLEDGGYTIYIKFAGKETPETPEE

SEQ ID NO: 15 - protein, 74 aa, synthetic construct
  REGION 1..74: note = Recombinantly produced
PKEEVTIKVHLIFADGKIQTAEFKGTFEEATAKAYAYADLLAKEHGEYTADLEDGGYTIQIKFAGKETPETPEE

SEQ ID NO: 16 - protein, 74 aa, synthetic construct
  REGION 1..74: note = Recombinantly produced
PKEEVTIKVHLIFADGKTQTAEFKGTFEEATAEAYRYADLLAKVHGEYTADLEDGGYTIYIKFAGKEQPGENPG

SEQ ID NO: 17 - protein, 74 aa, synthetic construct
  REGION 1..74: note = Recombinantly produced
PKEEVTIKVHLIFADGKTQTAEFKGTFEEATAEAYRYADLLAKVHGEYTADLEDGGYTIQIKFAGKEQPGENPG

SEQ ID NO: 18 - protein, 72 aa, synthetic construct
  REGION 1..72: note = Recombinantly produced
PKEEVTIKAHLIFADGKTQTAEFKGTFEEATAEAYRYADLLAKVHGEYTADLEDGGYTIYIKFAGKEQPGEN

SEQ ID NO: 19 - protein, 72 aa, synthetic construct
  REGION 1..72: note = Recombinantly produced
PKEEVTIKAHLIFADGKTQTAEFKGTFEEATAEAYRYADLLAKVHGEYTADLEDGGYTIQIKFAGKEQPGEN

SEQ ID NO: 20 - protein, 61 aa, synthetic construct
  REGION 1..61: note = Recombinantly produced
VTIKAHLIFANGSTQTAEFKGTFEKATSEAYAYADTLKKDHGEYTVDVADKGYTLYIKFAG

SEQ ID NO: 21 - protein, 61 aa, synthetic construct
  REGION 1..61: note = Recombinantly produced
VTIKAHLIFANGSTQTAEFKGTFEKATSEAYAYADTLKKDHGEYTVDVADKGYTLQIKFAG

SEQ ID NO: 22 - protein, 61 aa, synthetic construct
  REGION 1..61: note = Recombinantly produced
VTIKAHLIYADGKTQTAEFKGTFEEAAAEAYRYADALKKDHGEYTVDVADKGYTLYIKFAG

SEQ ID NO: 23 - protein, 61 aa, synthetic construct
  REGION 1..61: note = Recombinantly produced
VTIKAHLIYADGKTQTAEFKGTFEEAAAEAYRYADALKKDHGEYTVDVADKGYTLQIKFAG

SEQ ID NO: 24 - protein, 61 aa, synthetic construct
  REGION 1..61: note = Recombinantly produced
VTIKAHLIYADGKTQTAEFKGTFEEATAEAYRYADLLAKEHGKYTVDVADKGYTLYIKFAG

SEQ ID NO: 25 - protein, 61 aa, synthetic construct
  REGION 1..61: note = Recombinantly produced
VTIKAHLIYADGKTQTAEFKGTFEEATAEAYRYADLLAKEHGKYTVDVADKGYTLQIKFAG

SEQ ID NO: 26 - protein, 61 aa, synthetic construct
  REGION 1..61: note = Recombinantly produced
VTIKAHLIYADGKTQTAEFKGTFAEATAEAYRYADLLAKEHGKYTADLEDGGYTIYIRFAG

SEQ ID NO: 27 - protein, 61 aa, synthetic construct
  REGION 1..61: note = Recombinantly produced
VTIKAHLIYADGKTQTAEFKGTFAEATAEAYRYADLLAKEHGKYTADLEDGGYTIQIRFAG

SEQ ID NO: 28 - protein, 61 aa, synthetic construct
  REGION 1..61: note = Recombinantly produced
VTIKEHIYFEDGTVQTATFKGTFAEATAEAYRYADLLSKEHGKYTADLEDGGYTIYIRFAG

SEQ ID NO: 29 - protein, 61 aa, synthetic construct
  REGION 1..61: note = Recombinantly produced
VTIKEHIYFEDGTVQTATFKGTFAEATAEAYRYADLLSKEHGKYTADLEDGGYTIQIRFAG

SEQ ID NO: 30 - protein, 61 aa, synthetic construct
  REGION 1..61: note = Recombinantly produced
VTIKVHLIFADGKTQTAEFKGTFEEATAKAYAYADLLAKEHGEYTADLEDGGYTIYIKFAG

SEQ ID NO: 31 - protein, 61 aa, synthetic construct
  REGION 1..61: note = Recombinantly produced
VTIKVHLIFADGKTQTAEFKGTFEEATAKAYAYADLLAKEHGEYTADLEDGGYTIQIKFAG

SEQ ID NO: 32 - protein, 61 aa, synthetic construct
  REGION 1..61: note = Recombinantly produced
VTIKVHLIFADGKIQTAEFKGTFEEATAKAYAYADLLAKEHGEYTADLEDGGYTIYIKFAG

SEQ ID NO: 33 - protein, 61 aa, synthetic construct
  REGION 1..61: note = Recombinantly produced
VTIKVHLIFADGKIQTAEFKGTFEEATAKAYAYADLLAKEHGEYTADLEDGGYTIQIKFAG

SEQ ID NO: 34 - protein, 61 aa, synthetic construct
  REGION 1..61: note = Recombinantly produced
VTIKVHLIFADGKTQTAEFKGTFEEATAEAYRYADLLAKVHGEYTADLEDGGYTIYIKFAG

SEQ ID NO: 35 - protein, 61 aa, synthetic construct
  REGION 1..61: note = Recombinantly produced
VTIKVHLIFADGKTQTAEFKGTFEEATAEAYRYADLLAKVHGEYTADLEDGGYTIQIKFAG

SEQ ID NO: 36 - protein, 61 aa, synthetic construct
  REGION 1..61: note = Recombinantly produced
VTIKAHLIFADGKTQTAEFKGTFEEATAEAYRYADLLAKVHGEYTADLEDGGYTIYIKFAG

SEQ ID NO: 37 - protein, 61 aa, synthetic construct
  REGION 1..61: note = Recombinantly produced
VTIKAHLIFADGKTQTAEFKGTFEEATAEAYRYADLLAKVHGEYTADLEDGGYTIQIKFAG